(19) United States
(12) Patent Application Publication
(10) Pub. No.: US 2026/0256793 A1
(43) Pub. Date: Sep. 3, 2026

(54) ALTERATION OF F-BOX AND WD REPEAT DOMAIN CONTAINING 7 (FBXW7) GENE AS A PREDICTIVE BIOMARKER FOR TREATING CANCER WITH WEE1 INHIBITORS

(71) Applicant: Zeno Management, Inc., San Diego, CA (US)

(72) Inventors: Jianhui Ma, San Diego, CA (US); Xiao Guo, San Diego, CA (US); Olivier Harismendy, San Diego, CA (US); Wen Liu, San Diego, CA (US); Fernando Donate, Santa Monica, CA (US); Petrus Rudolf de Jong, San Diego, CA (US); Laure Escoubet, San Diego, CA (US)

(21) Appl. No.: 19/677,413

(22) Filed: May 14, 2026

Related U.S. Application Data
(63) Continuation of application No. PCT/US2024/055866, filed on Nov. 14, 2024.
(60) Provisional application No. 63/599,325, filed on Nov. 15, 2023.

(51) Int. Cl. A61K 31/519; A61K 31/506; A61P 35/00; C07K 16/28

(52) U.S. Cl. CPC A61K 31/519; A61K 31/506; A61P 35/00; C07K 16/2863

(57) ABSTRACT

The present disclosure provides, among other things, methods for treating cancer comprising administering an effective dose of a WEE1 inhibitor such as Azenosertib, or a pharmaceutically acceptable salt thereof, to a subject selected to have a cancer associated with at least one alteration in the F-box and WD repeat domain containing 7 (FBXW7) gene.

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6, including U.S. Provisional Application No. 63/599,325, filed Nov. 15, 2023, which is incorporated by reference in its entirety. The present application is a continuation of PCT Application No. PCT/US2024/055866, filed Nov. 14, 2024, which claims priority to U.S. Provisional Application No. 63/599,325, filed Nov. 15, 2023, each of which are incorporated by reference in their entireties including any drawings.

### REFERENCE TO SEQUENCE LISTING

The present application is filed with a Sequence Listing in Electronic format. The Sequence Listing is provided as a file entitled ZENO171C1.xml, created May 12, 2026, which is approximately 2,747 bytes in size. The information in the electronic format of the sequence listing is incorporated herein by reference in its entirety.

### BACKGROUND

DNA damage is typically resolved by a variety of pathways and proteins that repair damaged DNA. Prominent among these pathways are proteins involved in homologous recombination, a type of DNA repair in which nucleotide sequences are exchanged between two similar or identical molecules of DNA and that usually results in error free repair of damaged DNA. However, incorrect replacement of nucleotides into DNA can cause mutations and other genetic alterations that may lead to cancer development and progression. Improper DNA repair can lead to cell death, tumor development and progression. Cell cycle checkpoints are important for proper DNA repair, ensuring that cells do not progress with cellular replication until their genomic integrity is restored. There remains a need for therapies capable of effectively and reliably treating cancers, including cancers with DNA repair deficiencies.

### SUMMARY

Provided herein is the discovery that WEE1 inhibitors (e.g., Azenosertib or ZN-c3) can effectively treat cancers associated with at least one alteration in the F-box and WD repeat domain containing 7 (FBXW7) gene and that the FBXW7 gene alteration status can be used as a predictive biomarker for treating such cancers with one or more WEE1 inhibitors. In other words, it is found that cancers that are associated with at least one alteration in the FBXW7 gene exhibit better best response to treatment with a WEE1 inhibitor in the clinic, as compared to cancers carrying a wild-type FBXW7 genes.

Accordingly, in a first aspect, provided herein is a method of treating a cancer comprising administering an effective dose of a WEE1 inhibitor to a subject selected to have a cancer associated with at least one alteration in the FBXW7 gene. In one embodiment, provided herein is use of a WEE1 inhibitor in the manufacture of a medicament for treating cancer in a subject with an effective dose of the WEE1 inhibitor, wherein the subject is/was/has been selected to have a cancer associated with at least one alteration in the FBXW7 gene. In one embodiment, provided herein is a WEE1 inhibitor for use in treating cancer in a subject with an effective dose of the WEE1 inhibitor, wherein the subject is/was/has been selected to have a cancer associated with at least one alteration in the FBXW7 gene.

In a second aspect, provided herein is a method of treating a cancer comprising administering an effective dose of a WEE1 inhibitor to a subject selected to have a cancer associated with at least one alteration in the FBXW7 gene, wherein the WEE1 inhibitor is Azenosertib or a pharmaceutically acceptable salt thereof and the cancer is selected from colorectal cancer, peritoneum cancer, ovarian cancer, and endometrial cancer. In one embodiment, provided herein is use of a WEE1 inhibitor in the manufacture of a medicament for treating cancer in a subject with an effective dose of the WEE1 inhibitor, wherein the subject is/was/has been selected to have a cancer associated with at least one alteration in the FBXW7 gene; and wherein the WEE1 inhibitor is Azenosertib or a pharmaceutically acceptable salt thereof and the cancer is selected from colorectal cancer, peritoneum cancer, ovarian cancer, and endometrial cancer. In one embodiment, provided herein is a WEE1 inhibitor for use in treating cancer in a subject with an effective dose of the WEE1 inhibitor, wherein the subject is/was/has been selected to have a cancer associated with at least one alteration in the FBXW7 gene; and wherein the WEE1 inhibitor is Azenosertib or a pharmaceutically acceptable salt thereof and the cancer is selected from colorectal cancer, peritoneum cancer, ovarian cancer, and endometrial cancer.

In a third aspect, provided herein is a method of treating a gynecologic malignancy comprising administering an effective dose of a WEE1 inhibitor (e.g., Azenosertib or a pharmaceutically acceptable salt thereof) to a subject selected to have a gynecologic malignancy associated with at least one alteration in the FBXW7 gene. In one embodiment, provided herein is use of a WEE1 inhibitor (e.g., Azenosertib or a pharmaceutically acceptable salt thereof) in the manufacture of a medicament for treating a gynecologic malignancy in a subject with an effective dose of the WEE1 inhibitor, wherein the subject is/was/has been selected to have a gynecologic malignancy associated with at least one alteration in the FBXW7 gene. In one embodiment, provided herein is a WEE1 inhibitor (e.g., Azenosertib or a pharmaceutically acceptable salt thereof) for use in treating cancer in a subject with an effective dose of the WEE1 inhibitor, wherein the subject is/was/has been selected to have a gynecologic malignancy associated with at least one alteration in the FBXW7 gene.

In a fourth aspect, provided herein is a method of treating colorectal cancer comprising administering an effective dose of a WEE1 inhibitor (e.g., Azenosertib or a pharmaceutically acceptable salt thereof) to a subject selected to have colorectal cancer associated with at least one alteration in the FBXW7 gene. In one embodiment, provided herein is use of a WEE1 inhibitor (e.g., Azenosertib or a pharmaceutically acceptable salt thereof) in the manufacture of a medicament for treating colorectal cancer in a subject with an effective dose of the WEE1 inhibitor, wherein the subject is/was/has been selected to have colorectal cancer associated with at least one alteration in the FBXW7 gene. In one embodiment, provided herein is a WEE1 inhibitor (e.g., Azenosertib or a pharmaceutically acceptable salt thereof) for use in treating colorectal cancer in a subject with an effective dose of the WEE1 inhibitor, wherein the subject is/was/has been selected to have colorectal cancer associated with at least one alteration in the FBXW7 gene.

In a fifth aspect, provided herein is a method of treating uterine serous carcinoma (USC) comprising administering an effective dose of a WEE1 inhibitor (e.g., Azenosertib or a pharmaceutically acceptable salt thereof) to a subject selected to have USC associated with at least one alteration in the FBXW7 gene. In one embodiment, provided herein is use of a WEE1 inhibitor (e.g., Azenosertib or a pharmaceutically acceptable salt thereof) in the manufacture of a medicament for treating USC in a subject with an effective dose of the WEE1 inhibitor, wherein the subject is/was/has been selected to have USC associated with at least one alteration in the FBXW7 gene. In one embodiment, provided herein is a WEE1 inhibitor (e.g., Azenosertib or a pharmaceutically acceptable salt thereof) for use in treating USC in a subject with an effective dose of the WEE1 inhibitor, wherein the subject is/was/has been selected to have USC associated with at least one alteration in the FBXW7 gene.

Other features, objects, and advantages are apparent in the detailed description that follows. It should be understood, however, that the detailed description, while indicating embodiments, is given by way of illustration only, not limitation. Various changes and modifications within the scope of the present disclosure will become apparent to those skilled in the art from the detailed description.

### BRIEF DESCRIPTION OF THE DRAWINGS

Drawings are for illustration purposes only and not for limitation.

FIG. 1 is a graph showing co-mutation and mutual mutational exclusivity analysis of 15 molecular alteration pairs in subjects with TP53-mutated uterine serous carcinoma (USC) from four different sources.

FIG. 2 shows oncoprint maps from AACR-GENIE (top, N=796) and TCGA (bottom, N=95) subjects with TP53-mutated USC for genes showing significant co-mutation and mutual mutational exclusivity patterns.

FIG. 3A is a table that divides the PI3K-FBXW7-PPP2R1A trio molecular combination into six distinct subtypes based on the mutational status of the FBXW7, PPP2R1A, and PI3K (PIK3CA or PIK3R1). FIG. 3B shows the prevalence analysis of each subtype defined in FIG. 3A and unclassified subjects in each dataset.

FIG. 4A is a graph showing fractions of subjects who are CN2-positive or CN2-negative across the six PI3K-FBXW7-PPP2R1A trio molecular subtypes as defined in FIG. 3A. FIG. 4B is a graph showing Z-scores associated with AKT signaling of the various molecular subtypes. FIG. 4C is a graph showing Z-scores associated with TSC1 levels of the various molecular subtypes.

FIGS. 5A-5D are graphs analyzing overall survival of subjects with USC who are HRRm or HRRwt, and who are CCNE1-amplified or non-CCNE1-amplified, with the overall survival measured since sample collection or since initiation of the Carboplatin treatment.

FIG. 6 is a graph showing the Cyclin E1 immunohistochemistry (IHC) H-scores of subjects with high-grade serous ovarian cancer (HGSOC) and TP53-mutated USC and with or without the CCNE1-amplified status.

FIG. 7A is a graph showing Z-scores associated with Cyclin E1 levels of subjects with TP53-mutated USC and who are CCNE1-amplified or non-CCNE1-amplified.

FIG. 7B is a graph showing Z-scores associated with Cyclin-dependent kinase 1 (CDK1) protein expression levels of subjects with TP53-mutated USC and who are CCNE1-amplified or non-CCNE1-amplified. FIG. 7C is a graph showing Z-scores associated with Wnt/β-catenin signaling of subjects with TP53-mutated USC and who are CCNE1-amplified or non-CCNE1-amplified.

FIG. 8 is a Western Blot image showing levels of FBXW7, WEE1 kinase, Cyclin E1, and other replication stress, DNA damage, apoptosis markers in DLD-1 parental and DLD-1 FBXW7-knockout colorectal cancer cells treated with DMSO (negative control) and 250 nM, 1 μM, and 10 UM of Azenosertib for 8 hours, 24 hours, or 48 hours.

FIG. 9A shows the distribution of best overall response in FBXW7-mutated or wild-type human subjects.

FIG. 9B is a waterfall plot of the tumor size change in FBXW7-mutated or wild-type human subjects.

### DETAILED DESCRIPTION

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, the term “about” has its usual meaning as understood by those skilled in the art and thus indicates that a value includes the inherent variation of error for the method being employed to determine a value, or the variation that exists among multiple determinations.

As used herein, the terms “function” and “functional” have their usual meaning as understood by those skilled in the art and thus refer to a biological, enzymatic, or therapeutic function.

As used herein, the term “endogenous” has its usual meaning as understood by those skilled in the art and thus refers to the native, or wild type property of a gene, protein, or cell. In some embodiments, the endogenous gene is the wild type sequence of said gene. In some embodiment, the endogenous protein is the wild type sequence of said protein. In some embodiments, the endogenous protein function is the wild type function and activity level of said protein. In some embodiments, the endogenous cell is the wild type cell.

As used herein, the term “alteration” when referring to a genetic alteration or molecular alteration encompasses genetic mutations as further defined below, gene amplifications (e.g., high copy number), changes to gene expression, and changes to gene function, promoter silencing, as compared to the gene in its native state.

The term “mutation” has its usual meaning as understood by those skilled in the art and refers to an alteration of genetic sequence, by nucleotide substitution, nucleotide insertion, and/or nucleotide deletion. In some embodiments, cells have multiple mutations. In some embodiments, mutations are in coding regions of the genome. Mutations can range in size from a single base-pair, to a large segment of the chromosome that includes multiple genes. In some embodiments, at least one mutation is silent. In some embodiments, the mutation may have no significant impact on gene expression or function. In some embodiments, at least one mutation has an impact on gene expression or function, such as gene amplification, overexpression, or enhanced copy number. In some embodiments, at least one mutation is silent (e.g., not changing the coding sequence). In some embodiments, at least one mutation has a small impact on protein expression or function. In some embodiments, at least one mutation has a moderate impact on protein expression or function. In some embodiments, at least one mutation has a large impact on protein expression or function. In some embodiments, at least one mutation prevents protein expression or function. Non-limiting examples of mutations include insertions, deletions, truncations, substitutions, duplications, translocations, and inversions. In some embodiments, mutations are “somatic,” or occurring in body cells and are not inheritable. In some embodiments, a subset of somatic cells in an organism have at least one mutation that other somatic cells do not have. In some embodiments, mutations are “germline,” or occurring in germ cells and are inheritable. Mutations can also be categorized as pathogenic or likely pathogenic, which means that such mutations are likely to cause disease, such as cancer.

As disclosed herein, mutations, including mutations in the FBXW7 gene, can be monitored through a variety of sequencing, expression, or functional assays. Non-limiting examples include DNA sequencing, RNA sequencing, DNA hybridization, protein sequencing, targeted genomic sequencing, whole exome sequencing, whole genome sequencing, ATAC-sequencing, Sanger sequencing, PCR, qPCR, RT-PCR, RT-qPCR, Next Generation Sequencing, protein truncation test, DNA microarrays, heteroduplex analysis, denaturing gradient gel electrophoresis, nucleotide sequencing, single strand conformational polymorphism, restriction enzyme digestion assay, fluorescence in situ hybridization (FISH), comparative genomic hybridization, restriction fragment length polymorphism, amplification refractory mutation system PCR, nested PCR, multiplex ligation-dependent probe amplification, single strand conformational polymorphism, and oligonucleotide ligation assay. Mutations can also be monitored through a variety of antibody-based methods using biological samples including, but are not limited to, Western blotting, fluorescence activated cell sorting, immunofluorescence, immunohistochemistry, immunocytochemistry, immunoprecipitation, enzyme-linked immunosorbent assay, radioimmunoassays, and electrochemiluminescence assays.

The term “cancer” is used herein in its usual biological sense and understood by those skilled in the art. Thus, it can include the cancer of any cell type, such as, but not limited to, glioblastoma, (GBM) astrocytoma, meningioma, craniopharyngioma, medulloblastoma, other brain cancers, head and neck cancer, leukemia, AML (Acute Myeloid Leukemia), CLL (Chronic lymphocytic leukemia), ALL (Acute Lymphocytic Leukemia), myelodysplastic syndromes (MDS), skin cancer, adrenal cancer, anal cancer, bile duct cancer, bladder cancer, bone cancer, breast cancer, cervical cancer, colorectal cancer (or colon cancer), endometrial cancer (or endometrium cancer), esophagus cancer, eye cancer, gallbladder cancer, gastric cancer, gastrointestinal cancer, Hodgkin lymphoma, Non-Hodgkin lymphoma, hematological tumor, head cancer, hematologic malignancy, Kaposi sarcoma, kidney cancer, laryngeal and hypopharyngeal cancer, liver cancer, lung cancer, non-small cell lung cancer (NSCLC), small cell, lymphoma, mesothelioma, melanoma, multiple myeloma, neuroblastoma, nasopharyngeal cancer, neck cancer, ovarian cancer, osteosarcoma, sarcomas, gastrointestinal stromal tumor (GIST), pancreatic cancer, pituitary cancer, prostate cancer, renal cancer, retinoblastoma, salivary gland cancer, skin cancer, stomach cancer, small intestine cancer, spleen cancer, sarcomas, testicular cancer, thymus cancer, thyroid cancer, uterine cancer, endometrial endometrioid adenocarcinoma, uterine sarcoma, uterine serous carcinoma (USC), uterine carcinosarcoma, vaginal cancer, vulvar cancer, Waldenstrom macroglobulinemia, Wilms tumor, solid tumor, liquid tumor, high-grade serous ovarian cancer (HGSOC), invasive breast cancer, Triple Negative Breast Cancer (TNBC), esophagogastric cancer, gastric cancer, esophageal cancer, pRCC, ccRCC, chromophobe RCC, head and neck cancer, adenoid cystic carcinoma (ACC), Diffuse large B cell lymphoma (DLBCL), non-Hodgkin lymphoma (NHL), Low-grade gliomas (LGGs), Pheochromocytoma and paraganglioma (PCPGs), cholangiocarcinoma, acute myeloid leukemia (AML), CLL (Chronic lymphocytic leukemia), ALL (Acute Lymphocytic Leukemia), myelodysplastic syndromes (MDS), thymona, BRAF mutant metastatic colorectal cancer, uveal melanoma, high-grade serous ovarian, fallopian tube cancer, primary peritoneal cancer, BRAF V600E-mutated colorectal cancer, platinum-sensitive ovarian cancer, poly(ADP-ribose) polymerase inhibitor (PARPi)-resistant ovarian cancer, platinum-resistant ovarian cancer, platinum-refractory ovarian cancer, advanced pancreatic adenocarcinoma, pancreatic ductal adenocarcinoma, neuroendocrine tumor, neuroendocrine prostate cancer, pancreatic neuroendocrine tumor, small cell lung cancer (SCLC), germ cell cancer, stromal cancer, fallopian tube cancer and peritoneum cancer (e.g., primary peritoneal cancer).

As used herein, the term “tumor” has its usual meaning as understood by those skilled in the art and refers to an abnormal growth of cells or tissue. In some embodiments, the tumor is benign. In some embodiments, the tumor is malignant. A tumor becomes a cancer when it metastasizes, or spreads to other areas of the body. The term “solid tumor” as used herein has its usual meaning as understood by those skilled in the art and refers to an abnormal mass of tissue that does not contain liquid areas or cysts. Non-limiting examples of solid tumors include sarcomas, carcinomas, and lymphomas. Many cancer tissues can form solid tumors, such as, but not limited to, breast cancer, brain cancer, lung cancer, liver cancer, stomach cancer, spleen cancer, colon cancer, renal cancer, pancreatic cancer, prostate cancer, uterine cancer, skin cancer, head cancer, neck cancer, sarcomas, neuroblastomas and/or ovarian cancer. The terms “cancer” and “tumor” may generally be used interchangeably unless the context clearly indicates that a more specific meaning is intended.

The terms “individual”, “subject”, or “patient” as used herein have their usual meaning as understood by those skilled in the art and thus includes a human or a non-human mammal. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea or pigs. In some embodiments, the subject can be human. In some embodiments, the subject can be a child and/or an infant. In other embodiments, the subject can be an adult.

The term “cancer treatment” as used herein has its usual meaning as understood by those skilled in the art and refers to a therapeutic modality (such as surgery and/or radiation) or an anti-cancer agent such as a small molecule, compound, protein, or other medicant that is used to treat, inhibit, or prevent cancer.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), a sulfuric acid, a nitric acid and a phosphoric acid (such as 2,3-dihydroxypropyl dihydrogen phosphate). Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluenesulfonic, trifluoroacetic, benzoic, salicylic, 2-oxopentanedioic or naphthalenesulfonic acid. Pharmaceutical salts can also be obtained by reacting a compound with a base to form a salt such as an ammonium salt, an alkali metal salt, such as a sodium, a potassium or a lithium salt, an alkaline earth metal salt, such as a calcium or a magnesium salt, a salt of a carbonate, a salt of a bicarbonate, a salt of organic bases such as dicyclohexylamine, N-methyl-D-glucamine, tris(hydroxymethyl)methylamine, C₁-C₇ alkylamine, cyclohexylamine, triethanolamine, ethylenediamine and salts with amino acids such as arginine and lysine.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including, but not limited to, hydrogen-1 (protium) and hydrogen-2 (deuterium). Thus, reference herein to a compound encompasses all potential isotopic forms unless the context clearly dictates otherwise.

It is understood that the compounds described herein include crystalline forms (also known as polymorphs, which include the different crystal packing arrangements of the same elemental composition of a compound), amorphous phases, salts, solvates and hydrates. In some embodiments, the compounds described herein exist in solvated forms with pharmaceutically acceptable solvents such as water, ethanol or the like. In other embodiments, the compounds described herein exist in unsolvated form. Solvates contain either stoichiometric or non-stoichiometric amounts of a solvent and may be formed during the process of crystallization with pharmaceutically acceptable solvents such as water, ethanol or the like. Hydrates are formed when the solvent is water or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof, and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a compound, composition or device, the term “comprising” means that the compound, composition or device includes at least the recited features or components but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

The term “platinum-resistant” refers to a cancer that responds at first to treatment with drugs that contain the metal platinum, but the cancer requires treatment again within a certain period of time. For example, a cancer that requires within less than 6 months of platinum-free interval (PFI) after treatment is considered platinum-resistant. Of this group, those subjects who progress while on platinum-based therapy are often referred to as having “platinum-refractory” cancer.

The term “platinum-sensitive” refers to a cancer that responds at first to treatment with drugs that contain the metal platinum, but the cancer requires treatment again within a certain period of time. For example, a cancer that requires within more than 6 months of platinum-free interval (PFI) after treatment is considered platinum-resistant.

The term “PARP inhibitor-resistant” refers to a cancer that fails to respond to treatment with a PARP inhibitor, including cancers that are BRCA1/2-mutated or BRCA1/2-deficient.

Various aspects are described in detail in the following sections. The use of sections is not meant to limit the present disclosure. Each section can apply to any aspect of present disclosure. In this application, the use of “or” means “and/or” unless stated otherwise.

### WEE1 Inhibitors and Azenosertib

The present disclosure provides, among other things, methods for treating a cancer in a subject selected to have a cancer associated with at least one alteration in the F-box and WD repeat domain containing 7 (FBXW7) gene. In one embodiment, the at least one alteration is a genetic mutation, a gene amplification or both. In one embodiment, the genetic mutation is selected from a nucleotide substitution, a nucleotide insertion and a nucleotide deletion.

The present disclosure further provides methods of treating a cancer comprising administering an effective dose of a WEE1 inhibitor to a subject selected to have a cancer (e.g., a tumor or a tumor sample) associated with at least one alteration in the FBXW7 gene.

The terms “WEE1 inhibition,” “WEE1 inhibitor” and similar terms as used herein refer to the inhibition of the activity or function of a WEE1 tyrosine kinase, e.g., by degrading WEE1 tyrosine kinase and/or by reducing the activity of WEE1 tyrosine kinase with regard to mediating phosphorylation of CDK1 or CDK2. A WEE1 inhibitor that functions by degrading WEE1 tyrosine kinase may be referred to herein as a WEE1 degrader. The term “WEE1 inhibitor” includes all pharmaceutically acceptable salts thereof.

WEE1 is a tyrosine kinase that is a critical component of the ATR-mediated G2 cell cycle checkpoint control that prevents entry into mitosis in response to cellular DNA damage. Although the primary target of WEE1 is CDK1, WEE1 activation can lead to the selective phosphorylation of CDK2, thereby regulating CDK2-cyclin A/E complexes which control the G1/S phase progression. WEE1 inhibitor sensitivity has been associated with high levels of Cyclin E1 expression, which may result from gene amplification of CCNE1 and/or the alteration of its ubiquitin ligase gene, FBXW7. While CCNE1 gene amplification also has been associated with WEE1 inhibitor sensitivity and not wanting to be bound by any theory, there has been no correlation made between FBXW7 gene alteration status and WEE1 inhibitor sensitivity. Inhibition of WEE1 can result in excessive replication activity, thereby, leading to replication catastrophe. WEE1 inhibition has the potential to sensitize tumors to induce tumor cell death.

In one aspect, provided herein are methods of treating a cancer comprising administering an effective dose of a WEE1 inhibitor to a subject selected to have a cancer associated with at least one alteration in the FBXW7 gene. Non-limiting examples of WEE1 inhibitors for use in the methods described herein include those described in the following patent publications: WO 2019/074979, WO 2020/210383, WO 2020/210375, WO 2020/210377, WO 2020/210380, WO 2020/210381, WO 2022/082174, U.S. 2022/0162229, U.S. 2022/0168313, U.S. 2022/0169646, U.S. 2022/0220115, U.S. Pat. No. 11,332,473, WO 2019/173082, WO 2019/011228, WO 2019/138227, WO 2018/162932, WO 2018/011570, WO 2018/011569, U.S. 2022/0194947, WO 2018/090939, U.S. 2019/0308984, U.S. 2020/0131192, WO 2021/073491, WO 2022/188802, U.S. Pat. Nos. 11,345,710, 11,345,711, WO 2015/092431, WO 2015/019037, WO 2014/167347, WO 2007/126122, WO 2011/034743, U.S. 2007/0254892, WO 2008/133866, U.S. 2016/0060258, WO 2019/085933, WO 2020/221358, EP 3712150, WO 2018/133829, WO 2021/047627, U.S. 2021/0403451, WO 2020/083404, U.S. 2022/0324848, WO 2019/037678, WO 2018/171633, WO 2019/165204, WO 2012/161812, WO 2013/012681, WO 2013/013031, WO 2013/059485, WO 2013/126656, U.S. 2012/0220572, U.S. 2013/0018045, KR 2016-035878, KR 2020-016567, WO 2018/056621, WO 2017/075629, WO 2019/169065, WO 2019/134539, WO 2020/028814, U.S. 2021/0309630, WO 2020/069105, WO 2020/192581, U.S. 2022/0194960, CN 114831993, CN 115073466, CN 111718348, WO 96/34867, WO 2008/153207, WO 2010/067888, WO 2009/054332, WO 2021/074251, CN 112142763, WO 2020/259724, U.S. 2022/0259210, WO 2019/096322, CN 112142747, CN 112142748, U.S. 2022/0324868, WO 2021/043152, WO 2021/254389, WO 2022/171088, WO 2022/171126, WO 2022/171128, WO 2022/174765, WO 2022/174796, CN 112442049, CN 114072411, CN 113402520, CN 113387962, KR 2022-081171, WO 2022/124748, WO 2022/155202, CN 114591334, and CN 115197221, each of which is hereby incorporated by reference in their entireties.

In some embodiments, the WEE1 inhibitor is selected from the group consisting of Azenosertib (ZN-c3), Adavosertib (AZD1775), SC0191, Debio0123, IMP7068, PD0166285, NUV-569, SGR-3515, SY-4835, SPH-6162 April-1051 (formerly ATRN-W1051), ACR-2316, and a pharmaceutically acceptable salt of any of the foregoing. In one embodiment, the WEE1 inhibitor is Azenosertib or a pharmaceutically acceptable salt thereof. In one embodiment, the WEE1 inhibitor is Adavosertib or a pharmaceutically acceptable salt thereof. In one embodiment, the WEE1 inhibitor is Debio0123 or a pharmaceutically acceptable salt thereof. In one embodiment, the WEE1 inhibitor is IMP7068 or a pharmaceutically acceptable salt thereof. In one embodiment, the WEE1 inhibitor is SGR-3515 or a pharmaceutically acceptable salt thereof. In one embodiment, the WEE1 inhibitor is APR-1051 (formerly ATRN-W1051) or a pharmaceutically acceptable salt thereof. In one embodiment, the WEE1 inhibitor is ACR-2316 or a pharmaceutically acceptable salt thereof.

In one embodiment, the WEE1 inhibitor is Azenosertib, also called ZN-c3, having the following structure:

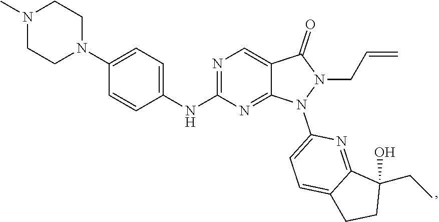 [SMILES: C=CCn1c(=O)c2cnc(Nc3ccc(N4CCN(C)CC4)cc3)nc2n1-c1ccc2c(n1)[C@@](O)(CC)CC2]

or a pharmaceutically acceptable salt thereof.

Accordingly, another aspect of this disclosure pertains to a method of treating a cancer comprising administering an effective dose of Azenosertib, or a pharmaceutically acceptable salt thereof, to a subject selected to have a cancer associated with at least one alteration in the FBXW7 gene.

Azenosertib and pharmaceutically acceptable salts thereof are WEE1 inhibitors. Azenosertib and pharmaceutically acceptable salts thereof can be prepared in various ways. See, e.g., WO 2019/173082, WO 2019/030401 and WO 2021/231653 describing Azenosertib, including methods of preparation and methods of treating cancer as a monotherapy or combination therapy with at least one second chemotherapeutic agent.

In other embodiments, the WEE1 inhibitor is the molecule as shown below:

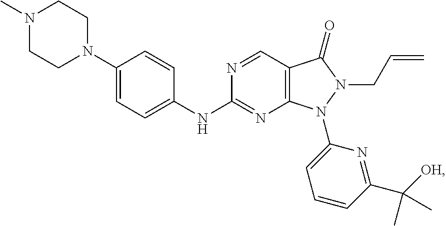 [SMILES: C=CCn1c(=O)c2cnc(Nc3ccc(N4CCN(C)CC4)cc3)nc2n1-c1cccc(C(C)(C)O)n1]

or a pharmaceutically acceptable salt thereof.

In other embodiments, the WEE1 inhibitor is the molecule as shown below:

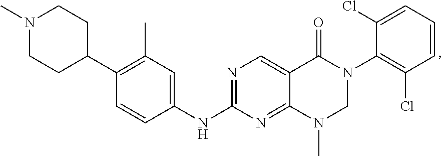 [SMILES: Cc1cc(Nc2ncc3c(n2)N(C)CN(c2c(Cl)cccc2Cl)C3=O)ccc1C1CCN(C)CC1]

or a pharmaceutically acceptable salt thereof or an N-oxide thereof.

In some embodiments, the WEE1 inhibitor is selected from the compounds below:

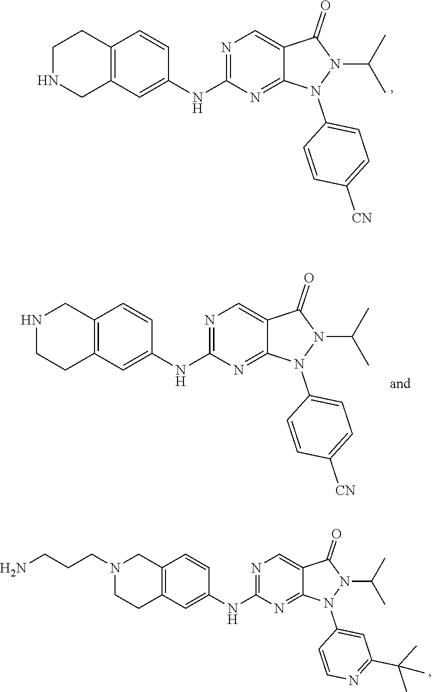

or a pharmaceutically acceptable salt of any of the foregoing.

In some embodiments, the WEE1 inhibitor is selected from the compounds below:

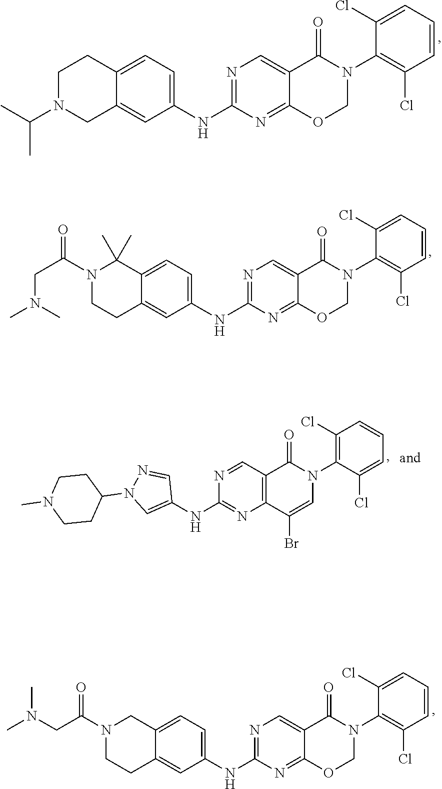 [SMILES: CC(C)N1CCc2ccc(Nc3ncc4c(n3)OCN(c3c(Cl)cccc3Cl)C4=O)cc2C1 ; CN(C)CC(=O)N1CCc2cc(Nc3ncc4c(n3)OCN(c3c(Cl)cccc3Cl)C4=O)ccc2C1(C)C ; CN1CCC(n2cc(Nc3ncc4c(=O)n(-c5c(Cl)cccc5Cl)cc(Br)c4n3)cn2)CC1 ; CN(C)CC(=O)N1CCc2cc(Nc3ncc4c(n3)OCN(c3c(Cl)cccc3Cl)C4=O)ccc2C1]

or a pharmaceutically acceptable salt of any of the foregoing.

In some embodiments, the WEE1 inhibitor is the compound below, or a pharmaceutically acceptable salt thereof:

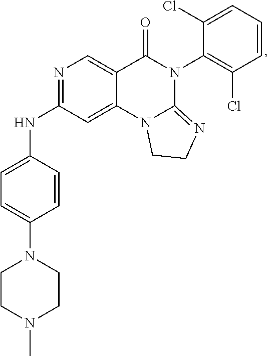 [SMILES: CN1CCN(c2ccc(Nc3cc4c(cn3)C(=O)N(c3c(Cl)cccc3Cl)C3=NCCN34)cc2)CC1]

or a pharmaceutically acceptable salt thereof.

In some embodiments, the WEE1 inhibitor is selected from the compounds below:

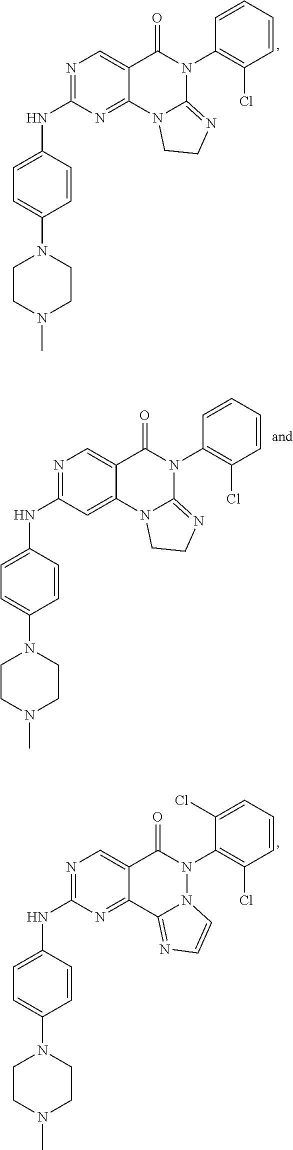 [SMILES: CN1CCN(c2ccc(Nc3ncc4c(n3)N3CCN=C3N(c3ccccc3Cl)C4=O)cc2)CC1 ; CN1CCN(c2ccc(Nc3cc4c(cn3)C(=O)N(c3ccccc3Cl)C3=NCCN34)cc2)CC1 ; CN1CCN(c2ccc(Nc3ncc4c(=O)n(-c5c(Cl)cccc5Cl)n5ccnc5c4n3)cc2)CC1]

or a pharmaceutically acceptable salt of any of the foregoing.

### The F-Box and WD Repeat Domain Containing 7 (FBXW7) Gene and Alterations of the Same

The gene encoding F-box protein FBXW7, FBXW7, is frequently genetically altered in many human cancers. In some embodiments, in a method described herein, the alteration(s) is selected from the group consisting of a genetic mutation, a genetic amplification, and a combination thereof. In one embodiment, the alteration(s) is a genetic mutation. In some embodiments, the genetic mutation is selected from nucleotide(s) substitution, a nucleotide(s) insertion, and nucleotide(s) deletion.

FBXW7 (also known as FBW7, Sel-10, hCdc4, or hAgo) is the F-box protein component of an Skp1-Cul1-F-box protein-type (SCF-type) ubiquitin ligase, in which it functions as a receptor responsible for substrate recognition. Most of the substrates of FBXW7 are growth promoters or proto-oncogenes, including cyclin E, c-Myc, myeloid cell leukemia-1 (Mcl-1), mammalian target of rapamycin (mTOR), Jun, Notch 1, Notch 2, Notch 3, Notch 4, and Aurora kinase A (AURKA), and FBXW7 is therefore thought to serve as a tumor suppressor of human cancers. FBXW7 expression increases following p53 activation, causes tumorigenesis in wild-type p53/FBXW7-mutated mice, and often demonstrates mutational and allelic loss in various human cancers.

In one embodiment, the FBXW7 protein has an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% similarity to SEQ ID NO:1:

(SEQ ID NO: 1)
MNQELLSVGSKRRRTGGSLRGNPSSSQVDEEQMNRVVEEEQQQQLRQQEEEHTARN
GEVVGVEPRPGGQNDSQQGQLEENNNRFISVDEDSSGNQEEQEEDEEHAGEQDEED
EEEEEMDQESDDFDQSDDSSREDEHTHTNSVTNSSSIVDLPVHQLSSPFYTKTTKMK
RKLDHGSEVRSFSLGKKPCKVSEYTSTTGLVPCSATPTTFGDLRAANGQGQQRRRIT
SVQPPTGLQEWLKMFQSWSGPEKLLALDELIDSCEPTQVKHMMQVIEPQFQRDFISL
LPKELALYVLSFLEPKDLLQAAQTCRYWRILAEDNLLWREKCKEEGIDEPLHIKRRK
VIKPGFIHSPWKSAYIRQHRIDTNWRRGELKSPKVLKGHDDHVITCLQFCGNRIVSGS
DDNTLKVWSAVTGKCLRTLVGHTGGVWSSQMRDNIIISGSTDRTLKVWNAETGECI
HTLYGHTSTVRCMHLHEKRVVSGSRDATLRVWDIETGQCLHVLMGHVAAVRCVQY
DGRRVVSGAYDFMVKVWDPETETCLHTLQGHTNRVYSLQFDGIHVVSGSLDTSIRV
WDVETGNCIHTLTGHQSLTSGMELKDNILVSGNADSTVKIWDIKTGQCLQTLQGPNK
HQSAVTCLQFNKNFVITSSDDGTVKLWDLKTGEFIRNLVTLESGGSGGVVWRIRASN
TKLVCAVGSRNGTEETKLLVLDFDVDMK.

Because the FBXW7 gene is a tumor suppressor gene, any mutation or alteration that leads to alteration to anywhere in the expressed protein or in splice sites or regulatory regions can potentially disrupt its associated function In some embodiments, in a method of treating a cancer described herein, the alteration(s) in the FBXW7 gene occurs at one or more nucleotides in a codon encoding an amino acid selected from the group consisting of E117, Q202, R224, R278, R367, G423, R441, R465, R479, R505, Y545, S582, R658, R668 and R689 in SEQ ID NO:1. In one embodiment, in a method of treating a cancer described herein, the alteration(s) in the FBXW7 gene results in an alternation selected from the group consisting of E117 deletion, Q220 frameshift mutation, (e.g., Q220fs*19), R465H, R479Q, R505C, R505G, R505L and R689W in SEQ ID NO:1; and splice-site mutation FBXW7_c.585-4_592delins.

In some embodiments, in a method described herein, the alteration(s) in the FBXW7 gene results in partial or complete loss of expression of the FBXW7 gene. In some embodiments, a partial loss of expression of the FBXW7 gene means a loss of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the expression of the FBXW7 gene as compared to an unaltered, wild-type or native FBXW7 gene, whether in an in vitro, in vivo pre-clinical, or clinical setting.

In some embodiments, in a method of treating a cancer described herein, the alteration(s) in the FBXW7 gene results in alterations in the cellular distribution of the expressed FBXW7 protein. In some embodiments, in a method of treating a cancer described herein, the alteration(s) in the FBXW7 gene results in partial or complete loss of the ability of the expressed FBXW7 protein to interact with and/or degrade one or more oncoproteins. In some embodiments, a partial loss of the ability of the expressed FBXW7 protein to interact with and/or degrade one or more oncoproteins means a loss of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the ability of the expressed FBXW7 protein to interact with and/or degrade one or more oncoproteins as compared to a FBXW7 expressed from an unaltered, wild-type or native FBXW7 gene, whether in an in vitro, in vivo pre-clinical, or clinical setting. In some embodiments, these one or more oncoproteins are any one or more oncoproteins selected from the group consisting of cyclin E, c-Myc, myeloid cell leukemia-1 (Mcl-1), mammalian target of rapamycin (mTOR), Jun, Notch 1, Notch 2, Notch 3, Notch 4, and Aurora kinase A (AURKA).

In some embodiments, in a method described herein, the expressed FBXW7 protein is an isoform selected from the group consisting of FBXW7α, FBXW7β, and FBXW7γ.

### Subject History, Optional Additional Selections, and Other Cancer Characterizations

In one embodiment, the method of treating a cancer described herein is where the cancer being treated is not associated with any oncogene alteration or status, any oncoprotein expression level or status, and/or any signaling pathway level or alteration, other than the at least one alteration in FBXW7.

In alternative embodiments, in a method of treating a cancer described herein, the subject is further selected to have a cancer associated with any oncogene alteration or status, any oncoprotein expression level or status, and/or any signaling pathway level or alteration, other than the at least one alteration in FBXW7.

In some embodiments, the subject selected to have a cancer associated with at least one alteration in FBXW7 and may be further characterized by certain other genomic instability, oncoprotein statuses, and/or signaling pathway levels or alterations as described herein.

In the embodiments described below, the term “the subject selected” refers to a subject selected to have a cancer associated with at least one alteration in FBXW7.

### TP53, PTEN, PPP2R1A, PIK3CA, PIK3R1, and KRAS

In some embodiments, in a method of treating cancer described herein, the subject is further selected to have a cancer associated with at least one other alteration in a gene selected from the group consisting of TP53, PTEN, PPP2R1A, PIK3CA, PIK3R1, and KRAS. In some embodiments, in a method of treating cancer described herein, the subject is further selected to have a cancer associated with at least one other alteration in a gene selected from the group consisting of TP53, PTEN, PIK3CA, PIK3R1, and KRAS. In one embodiment, in a method of treating cancer described herein, the subject is further selected to have a cancer associated with at least one other alteration in TP53. In one embodiment, in a method of treating cancer described herein, the subject is further selected to have a cancer associated with at least one other alteration in one or both of PIK3CA and PIK3R1. In one embodiment, in a method of treating cancer described herein, the subject is further selected to have a cancer associated with at least one other alteration in PTEN and one or both of PIK3CA and PIK3R1. In one embodiment, in a method of treating cancer described herein, the subject is further selected to have a cancer associated with at least one other alteration in PIK3CA but not PIK3R1. In one embodiment, in a method of treating cancer described herein, the subject is further selected to have a cancer associated with at least one other alteration in PTEN and PIK3CA but not PIK3R1. In one embodiment, in a method of treating cancer described herein, the subject is further selected to have a cancer associated with at least one other alteration in PIK3R1 but not PIK3CA. In one embodiment, in a method of treating cancer described herein, the subject is further selected to have a cancer associated with at least one other alteration in PTEN and PIK3R1 but not PIK3CA.

In some embodiments, in a method of treating cancer described herein, the subject selected has a cancer associated with at least one other alteration in a gene selected from the group consisting of TP53, PTEN, PPP2R1A, PIK3CA, PIK3R1, and KRAS. In other embodiments, in a method of treating cancer described herein, the subject selected does not have a cancer associated with at least one other alteration in a gene selected from the group consisting of TP53, PTEN, PPP2R1A, PIK3CA, PIK3R1, and KRAS. In some embodiments, in a method of treating cancer described herein, the subject selected has a cancer associated with at least one other alteration in a gene selected from the group consisting of TP53, PTEN, PIK3CA, PIK3R1, and KRAS. In other embodiments, in a method of treating cancer described herein, the subject selected does not have a cancer associated with at least one other alteration in a gene selected from the group consisting of TP53, PTEN, PIK3CA, PIK3R1, and KRAS. In one embodiment, in a method of treating cancer described herein, the subject selected has a cancer associated with at least one other alteration in TP53. In another embodiment, in a method of treating cancer described herein, the subject selected does not have a cancer associated with at least one other alteration in TP53. In one embodiment, in a method of treating cancer described herein, the subject selected has a cancer associated with at least one other alteration in PPP2R1A. In another embodiment, in a method of treating cancer described herein, the subject selected does not have a cancer associated with at least one other alteration in PPP2R1A. In one embodiment, in a method of treating cancer described herein, the subject selected has a cancer associated with at least one other alteration in one or both of PIK3CA and PIK3R1. In one embodiment, in a method of treating cancer described herein, the subject selected has a cancer associated with at least one other alteration in PTEN and one or both of PIK3CA and PIK3R1. In another embodiment, in a method of treating cancer described herein, the subject selected does not have a cancer associated with at least one other alteration in one or both of PIK3CA and PIK3R1. In another embodiment, in a method of treating cancer described herein, the subject selected does not have a cancer associated with at least one other alteration in PTEN and one or both of PIK3CA and PIK3R1. In one embodiment, in a method of treating cancer described herein, the subject selected has a cancer associated with at least one other alteration in PIK3CA but not PIK3R1. In one embodiment, in a method of treating cancer described herein, the subject selected has a cancer associated with at least one other alteration in PTEN and PIK3CA but not PIK3R1. In one embodiment, in a method of treating cancer described herein, the subject selected has a cancer associated with at least one other alteration in PIK3R1 but not PIK3CA. In one embodiment, in a method of treating cancer described herein, the subject selected has a cancer associated with at least one other alteration in PTEN and PIK3R1 but not PIK3CA. In another embodiment, in a method of treating cancer described herein, the subject selected does not have a cancer associated with an alteration in either of PIK3CA and PIK3R1. In another embodiment, in a method of treating cancer described herein, the subject selected does not have a cancer associated with an alteration in PTEN and either of PIK3CA and PIK3R1.

### APC and ARID1A

In some embodiments, in a method of treating cancer described herein, the subject is further selected to have a cancer associated with at least one other alteration in a gene selected from the group consisting of APC and ARID1A. In some embodiments, in a method of treating cancer described herein, the subject selected has a cancer associated with at least one other alteration in a gene selected from the group consisting of APC and ARID1A. In other embodiments, in a method of treating cancer described herein, the subject selected does not have a cancer associated with at least one other alteration in a gene selected from the group consisting of APC and ARID1A.

### CCNE1, MYC, and ERBB2

In some embodiments, in a method of treating cancer described herein, the subject is further selected to have a cancer associated with amplification or a high copy number of a gene selected from the group consisting of CCNE1, MYC, and ERBB2. In one embodiment, the amplified status or a high copy number status of any one of the genes CCNE1, MYC, and ERBB2 means an average of median gene copy number of at least about 3, at least about 4, at least about 5, at least about 6, at least about 7, or at least about 8. In one embodiment, the subject is further selected to have a cancer associated with amplification or a high copy number of CCNE1. In one embodiment, the subject selected has a cancer associated with amplification or a high copy number of CCNE1. In another embodiment, the subject selected does not have a cancer associated with amplification or a high copy number of CCNE1.

### Cyclin E1, CDK1, and Wnt/β-Catenin Signaling

In some embodiments, in a method of treating cancer described herein, the subject is further selected to have a cancer associated with high or increased levels of Cyclin E1 protein expression. In some embodiments, in a method of treating cancer described herein, the subject further selected has a cancer associated with high or increased levels of Cyclin E1 protein expression. In other embodiments, in a method of treating cancer described herein, the subject further selected does not have a cancer associated with high or increased levels of Cyclin E1 protein expression.

In some embodiments, high or increased levels of gene expression, protein expression, or pathway signaling means that, compared to a reference individual or a population of individuals with no cancer, the level of gene expression, protein expression, or pathway signaling is increased by at least about at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

In some embodiments, low or reduced levels of gene expression, protein expression, or pathway signaling means that, compared to a reference individual or a population of individuals with no cancer, the level of gene expression, protein expression, or pathway signaling is reduced by at least about at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

Protein expression levels, such as Cyclin E1 expression levels, may be measured, for example, by CCNE1 mRNA or transcript levels or by immunohistochemistry (IHC) and the expression levels may be expressed as IHC H-scores. High expression levels of Cyclin E1 may be determined by certain H-score cut-offs (e.g., >50, >125), or other cut-offs such as certain percentages (e.g., >10%, >30%) of viable tumor cells having an IHC staining intensity of 1+, 2+, or 3+, and may be assigned status such as Cyclin E1-positive, Cyclin E1-positive (low), Cyclin E1-positive (high), and Cyclin E1-high.

In some embodiments, in a method of treating cancer described herein, the subject is further selected to have a cancer associated with high or increased levels of Cyclin-dependent kinase 1 (CDK1) protein expression. In some embodiments, in a method of treating cancer described herein, the subject further selected has a cancer associated with high or increased levels of CDK1 protein expression. In other embodiments, in a method of treating cancer described herein, the subject further selected does not have a cancer associated with high or increased levels of CDK1 protein expression.

In some embodiments, in a method of treating cancer described herein, the subject is further selected to have a cancer associated with low or reduced Wnt/β-catenin signaling. In some embodiments, in a method of treating cancer described herein, the subject further selected has a cancer associated with low or reduced Wnt/β-catenin signaling. In other embodiments, in a method of treating cancer described herein, the subject further selected does not have a cancer associated with low or reduced Wnt/β-catenin signaling.

### Homologous Recombination Repair (HRR) Genes and HRR Statuses

In some embodiments, in a method of treating cancer described herein, the subject is further selected to have a cancer associated with at least one other alteration in a homologous recombination and repair (HRR) gene. In some embodiments, in a method of treating cancer described herein, the subject selected has a cancer associated with at least one other alteration in a homologous recombination and repair (HRR) gene. In some embodiments, in a method of treating cancer described herein, the subject is selected does not have a cancer associated with at least one other alteration in a homologous recombination and repair (HRR) gene.

In some embodiments, the HRR gene is any one or more genes selected from the group consisting of BRCA1, BRCA2, ATM, CHEK1, CHEK2, FANCL, CDK12, PALB2, BRIP1, BARD1, RAD51B, RAD51C, RAD51D, and RAD54L. In some embodiments, in a method of treating cancer described herein, the subject is further selected to have a cancer associated with at least one other alteration in BRCA1. In some embodiments, in a method of treating cancer described herein, the subject is further selected to have a cancer associated with at least one other alteration in BRCA1. In one embodiment, a cancer or a subject with a cancer associated with at least one other alteration in any one of these HRR genes is given a status of “HRR-mutated” or “HRRm” while a cancer or a subject with a cancer not associated with at least one other alteration in any one of these HRR genes is given a status of “HRR-wild type” or “HRRwt.”

HRRm status may be determined by an assay such as e.g., FoundationOne® CDx, Myriad (MyChoice® CDx), Tempus xT HRD, Caris Molecular. Intelligence Comprehensive Tumor Profiling or any other CLIA certified (or local equivalent) lab may also be used. Confirmed deleterious mutations in at least 1 of the genes involved in HRR as determined from CLIA-approved (or country-specific equivalent) prior genomic profiling.

### Homologous Recombination Deficiency (HRD) Statuses

In some embodiments, in a method of treating cancer described herein, the subject is further selected to have a cancer associated with a homologous recombination deficiency (HRD) status. HRD status may be due to a point mutation as described above or other genetic changes such as copy number variation, somatic copy number alterations (SCNA), aneuploidy, loss of heterozygosity (LOH), large-scale transition (LST), and/or telomeric allelic imbalance (TAI). In some embodiments, the subject has HRD due to a copy number variation, somatic copy number alterations (SCNA), aneuploidy, loss of heterozygosity (LOH), large-scale transition (LST), and/or telomeric allelic imbalance (TAI). In some embodiments, the subject has HRD due to a copy number variation. In some embodiments, the subject has HRD due to a somatic copy number alterations (SCNA). In some embodiments, the subject has HRD due to an aneuploidy. In some embodiments, the subject has HRD due to a loss of heterozygosity (LOH). In some embodiments, the subject has HRD due to a large-scale transition (LST). In some embodiments, the subject has HRD due to a telomeric allelic imbalance (TAI).

Subject selection or cancer type characterization may be based on HRD biomarker status using a variety of methods known in the art. In some embodiments, HRD is characterized by a deleterious or suspected deleterious BRCA mutation and/or genomic instability. In some embodiments, HRD is characterized by a deleterious or suspected deleterious BRCA mutation. In some embodiments, HRD is characterized by a genomic instability.

In some embodiments, HRD status is assessed by the mutation status of BRCA1/2 and/or specific patterns of genomic instability—measured by the evaluation of a combination of one or more genome-wide measures to derive a HRD related genomic instability score (e.g., gLOH+TAI+LST). In some embodiments, HRD status is defined by the mutation status of BRCA1/2 and/or genomic instability as measured by gLOH. In some embodiments, the HRD status is an absolute value or standard. In some embodiments, the HRD status is determined based on whether the subject has a gene alteration in one or more genes in the HR pathway. In some embodiments, HRD status is determined using information derived from RNA sequencing and DNA sequencing of cancerous tissue. In some embodiments, DNA sequencing data from matched cancerous tissue and germline tissue are used together to determine HRD status. In some embodiments, HRD status is determined based on predictions using RNA or DNA sequencing information. In some embodiments, HRD status is determined using mRNA transcription data generated from a cancerous tissue or a subject. In some embodiments, HRD status is determined using genome-wide loss of heterozygosity (gwLOH) determined from DNA sequencing data generated from a cancerous tissue. In some embodiments, HRD status is determined using DNA sequencing from a matched non-cancerous tissue. In some embodiments, HRD status is determined using both RNA and DNA sequencing data.

In some embodiments, HRD-negative status is classified as a genetic profile with no single nucleotide variants, no short insertions or deletions, and diploid copy number of BRCA1 and BRCA2 genes.

In some embodiments, HRD-positive status is classified as including one or more features of the RNA and/or DNA sequencing data (e.g., one or more of mRNA expression levels for a plurality of genes, a measure of loss of genomic heterozygosity, a measure of genomic and/or transcriptomic rearrangements (e.g., one or more of insertions, deletions, gene fusions, inversions, etc.), a measure of genomic methylation, etc.).

In some embodiments, testing for HRD variants is based known pathogenic variants in the germline sequence of one or more HR associated genes (e.g., BRCA1, BRCA2, ATM, CHEK1, CHEK2, FANCL, CDK12, PALB2, BRIP1, BARD1, RAD51B, RAD51C, RAD51D, and/or RAD54L).

In some embodiments, HRD status is determined by a diagnostic assay. In some embodiments, HRD status is determined using a genotypic assay. In some embodiments, HRD status is determined using a phenotypic assay. HRD status can be determined using any method known in the art. For example, exemplary methods are described in Stewart et al., Oncologist (2022 Mar. 11) 27 (3): 167-174. (PMID: 35274707; PMCID: PMC8914493), which is hereby incorporated by reference in its entirety.

In some embodiments, the diagnostic test is a molecular (sequence-) based in vitro diagnostic test for the HRD status of a cancer. Clinical-grade HRD assays detecting ‘genomic scars’ ‘Genomic scars’ serve may be used as a measure of HRD. An HRD genomic scar assay evaluates for the percentage of genomic regions with LOH determined through tumor single-nucleotide polymorphism (SNP) sequencing (FoundationOne CDx, Foundation Medicine) or through a genomic instability score (GIS) calculated by combining three factors obtained from allele-specific copy number profiles for SNP-LOH, telomeric allelic imbalance (TAI), and large-scale transitions (LSTs) (myChoice CDx, Myriad Genetics).

In some embodiments, the HRD diagnostic assay is a companion diagnostic used for treatment with a PARP inhibitor. For example, in some embodiments, the diagnostic test is a molecular (sequence-) based in vitro diagnostic test (“MyChoice”) for the HRD status of a cancer available from Myriad Genetics, Inc. This test is approved by the FDA (USA) as a companion diagnostic for use of the PARP inhibitor niraparib.

In some embodiments, HRD is defined by tumor BRCA mutation or a composite genomic instability score of greater than or equal to 42 (a cancer is characterized as HRD if the test score (HRD score) is at least 42, otherwise it is characterized as not-HRD (HRP).

In some embodiments, HRD score is determined by BRCA1/2 mutation status. In some embodiments, BRCA1/2 mutation is denoted as homologous recombination deficiency (HRD) positivity. In some embodiments, the diagnostic method is the “MyChoice” diagnostic test of Myriad Genetics, Inc. In some embodiments, HRD status is based on a predetermined threshold. In some embodiments, the threshold is a test score (HRD score) of 42 for characterizing (or classifying) a cancer as HRD or HRP (not-HRD).

In some embodiments, the diagnostic method includes the % LOH score measuring the percentage of genomic LOH as a marker of HRD positivity, % LOH≥16 is denoted as % LOH-high. In some embodiments, the diagnostic method is the FoundationOne CDx (Foundation Medicine). In some embodiments, the FoundationOne CDx (Foundation Medicine) and includes the % LOH score measuring the percentage of genomic LOH as a marker of HRD positivity, % LOH≥16 is denoted as % LOH-high.

HRD-positive status may be classified using an HRD score. HRD score may be determined by considering one or more features of the RNA and/or DNA sequencing data (e.g., one or more of mRNA expression levels for a plurality of genes, a measure of loss of genomic heterozygosity, a measure of genomic and/or transcriptomic rearrangements (e.g., one or more of insertions, deletions, gene fusions, inversions, etc.), a measure of genomic methylation, etc.). Exemplary methods for determining HRD score are described for example in Lotan, T. L., et. al. Mod Pathol 34, 1185-1193 (2021), which is hereby incorporated by reference in its entirety.

In some embodiments, HRD score may be determined using one or more features associated with homologous recombination deficiency. In some embodiments, such features comprise one or more of a mutation score associated with the cancer, a telomeric allelic imbalance score, a large-scale state transition (LST) score associated with the cancer, a loss of heterozygosity (LOH) score, a fraction of the genome having lost heterogeneity (fLOH), or a homologous recombination deficiency score (the sum of one or more of the telomeric allelic imbalance (NtAI) score, the large-scale state transition (LST) score, fraction of genome having lost heterogeneity (fLOH), and/or the loss of heterozygosity (LOH) score). In some embodiments, the homologous recombination deficiency (HRD) score is a sum of NtAI, LST, and LOH.

The number of telomeric allelic imbalances (NtAI) score relates the number of subtelomeric regions with allelic imbalance that start from beyond the centromere and extend to the telomere. The largest-scale state transitions (LST) score relates to the number of large chromosomal breaks between adjacent regions, generally of at least about 10 megabases (Mb), although the specific threshold size may be increased or decreased. The loss of heterozygosity score (HRD-LOH) relates to the number of regions with a loss of heterozygosity, generally larger than 15 Mb (although the specific threshold size may be increased or decreased), but shorter than the whole chromosome. In some embodiments, the features may include one or more of NtAI, LST, LOH, or an HRD score (the sum of one or more of NtAI, LST and LOH).

In some embodiments, the one or more features comprise LST. In some embodiments, the one or more features do not include NtAI. In some embodiments, the one or more features do not include LOH. In some embodiments, the one or more features do not include NtAI and LOH. In some embodiments, HRD score, NtAI score, LST score, and/or LOH score is determined by a microarray or by sequencing (for example, whole exome sequencing or whole genome sequencing) nucleic acids derived from the cancer. In some embodiments, fraction of the genome with a loss of heterogeneity (fLOH) may be used as a feature.

### Subject History and Prior Treatments

In some embodiments, in a method of treating cancer described herein, the subject has received no more than 1, at least 1, 1, 2, 3, 4, 1 or 2, 1 to 2, 1 to 3, or 1 to 4 prior line(s) of therapy, prior line(s) of therapy in the advanced or metastatic setting, prior line(s) of chemotherapy, prior line(s) of platinum-based chemotherapy, prior regimen(s), or prior therapeutic regimen(s). Such cancers that have been previously treated can be assigned certain sensitivity, resistance, and refractory statuses, which are further described below.

### Methods of Treatment

In some embodiments, a method of treating a cancer described herein results in a therapeutic effect (e.g., a desired pharmacologic and/or physiologic effect). A therapeutic effect can encompass partially or completely curing a cancer, relieving one or more adverse symptoms attributable to the cancer, delaying or attenuating progression of the cancer, maintaining regression, preventing or delaying relapse of the cancer, etc. To this end, the method comprises administering an effective dose or a therapeutically effective amount of Azenosertib, or a pharmaceutically acceptable salt thereof, without in combination or in combination with at least one a second chemotherapeutic agent, or a pharmaceutically acceptable salt thereof. A therapeutically effective amount can be an amount effective, at dosages and for periods of time necessary, to achieve a desired therapeutic result (e.g., tumor growth inhibition, progression free survival, complete response, partial response etc.). A therapeutically effective amount may vary according to factors such as the disease state, age, sex, and weight of the individual, and the ability of the binding agent to elicit a desired response in the individual.

In some embodiments, therapeutic response may be determined according to Response Evaluation Criteria in Solid Tumors (RECIST) criteria.

### Cancer Types

Methods of the present disclosure can be used to treat a variety of cancers.

In some embodiments, the cancer is selected from glioblastoma, (GBM) astrocytoma, meningioma, craniopharyngioma, medulloblastoma, other brain cancers, head and neck cancer, leukemia, AML (Acute Myeloid Leukemia), CLL (Chronic lymphocytic leukemia), ALL (Acute Lymphocytic Leukemia), myelodysplastic syndromes (MDS), skin cancer, adrenal cancer, anal cancer, bile duct cancer, bladder cancer, bone cancer, breast cancer, cervical cancer, colorectal cancer (or colon cancer), endometrial cancer (or endometrium cancer), esophagus cancer, eye cancer, gallbladder cancer, gastric cancer, gastrointestinal cancer, Hodgkin lymphoma, Non-Hodgkin lymphoma, hematological tumor, head cancer, hematologic malignancy, Kaposi sarcoma, kidney cancer, laryngeal and hypopharyngeal cancer, liver cancer, lung cancer, non-small cell lung cancer (NSCLC), small cell, lymphoma, mesothelioma, melanoma, multiple myeloma, neuroblastoma, nasopharyngeal cancer, neck cancer, ovarian cancer, osteosarcoma, sarcomas, gastrointestinal stromal tumor (GIST), pancreatic cancer, pituitary cancer, prostate cancer, renal cancer, retinoblastoma, salivary gland cancer, skin cancer, stomach cancer, small intestine cancer, spleen cancer, sarcomas, testicular cancer, thymus cancer, thyroid cancer, uterine cancer, endometrial endometrioid adenocarcinoma, uterine sarcoma, uterine serous carcinoma (USC), uterine carcinosarcoma, vaginal cancer, vulvar cancer, Waldenstrom macroglobulinemia, Wilms tumor, solid tumor, liquid tumor, high-grade serous ovarian cancer (HGSOC), invasive breast cancer, Triple Negative Breast Cancer (TNBC), esophagogastric cancer, gastric cancer, esophageal cancer, pRCC, ccRCC, chromophobe RCC, head and neck cancer, adenoid cystic carcinoma (ACC), Diffuse large B cell lymphoma (DLBCL), non-Hodgkin lymphoma (NHL), Low-grade gliomas (LGGs), Pheochromocytoma and paraganglioma (PCPGs), cholangiocarcinoma, acute myeloid leukemia (AML), CLL (Chronic lymphocytic leukemia), ALL (Acute Lymphocytic Leukemia), myelodysplastic syndromes (MDS), thymona, BRAF mutant metastatic colorectal cancer, uveal melanoma, high-grade serous ovarian, fallopian tube cancer, primary peritoneal cancer, BRAF V600E-mutated colorectal cancer, platinum-sensitive ovarian cancer, poly(ADP-ribose) polymerase inhibitor (PARPi)-resistant ovarian cancer, platinum-resistant ovarian cancer, platinum-refractory ovarian cancer, advanced pancreatic adenocarcinoma, pancreatic ductal adenocarcinoma, neuroendocrine tumor, neuroendocrine prostate cancer, pancreatic neuroendocrine tumor, small cell lung cancer (SCLC), germ cell cancer, stromal cancer, fallopian tube cancer and peritoneum cancer (e.g., primary peritoneal cancer).

In some embodiments, the cancer is selected from colorectal cancer, peritoneum cancer, ovarian cancer and uterine cancer. In some embodiments, the cancer is selected from colorectal cancer, peritoneum cancer and a gynecologic malignancy. In some embodiments, the cancer is a gynecologic malignancy. In some embodiments, the cancer or the gynecologic malignancy is selected from cervical cancer, ovarian cancer, uterine cancer, vaginal cancer, vulvar cancer and fallopian tube cancer.

In one embodiment, the cancer or the gynecologic malignancy is uterine cancer. In one embodiment, the cancer or the gynecologic malignancy is selected from endometrial cancer, endometrial endometrioid adenocarcinoma, uterine sarcoma, uterine serous carcinoma (USC) and uterine carcinosarcoma. In one embodiment, the cancer or the gynecologic malignancy is endometrial cancer. In one embodiment, the cancer or the gynecologic malignancy is uterine serous carcinoma (USC) (e.g., TP53-mutated USC).

In one embodiment, the cancer or the gynecologic malignancy is ovarian cancer. In one embodiment, the cancer or the gynecologic malignancy is selected from high-grade serous ovarian cancer (HGSOC), platinum-sensitive ovarian cancer, poly(ADP-ribose) polymerase inhibitor (PARPi)-resistant ovarian cancer, platinum-resistant ovarian cancer and platinum-refractory ovarian cancer. In one embodiment, the cancer or the gynecologic malignancy is platinum-refractory ovarian cancer. In one embodiment, the cancer or the gynecologic malignancy is high-grade serous ovarian cancer (HGSOC).

In one embodiment, the cancer is colorectal cancer. In one embodiment, the cancer is peritoneum cancer. In one embodiment, the cancer is TP53-mutated USC.

In one embodiment, the methods described herein comprise administering the effective dose of a WEE1 inhibitor as monotherapy (i.e., without being in combination with any other therapeutic agent). In an alternative embodiment, the methods described herein comprise administering the effective dose of a WEE1 inhibitor in combination with encorafenib and optionally further in combination with cetuximab, including but not limited to, for treatment of colorectal cancer.

In some embodiments, the cancer is histologically or cytologically confirmed. In the some embodiments, the cancer is pathologically confirmed. In some embodiments, the cancer is recurrent or persistent. In some embodiments, the cancer is metastatic. In some embodiments, the cancer is unresectable.

In some embodiments, the cancer is cancer is platinum-resistant, platinum-sensitive, or platinum-refractory. In some embodiments, the cancer is PARP inhibitor-resistant.

### Effective Doses and Dosing Schedules

As used herein, the terms “treat,” “treating,” “treatment,” “therapeutic,” and “therapy” do not necessarily mean total cure or abolition of the disease and/or condition, such as osteosarcoma. Any alleviation of any undesired signs or symptoms of the disease and/or condition (such as osteosarcoma), to any extent can be considered treatment and/or therapy. Furthermore, treatment may include acts that may worsen the subject's overall feeling of well-being or appearance.

The term “effective dose” is used to indicate an amount of a WEE1 inhibitor (including pharmaceutically acceptable salts thereof) (e.g., Azenosertib or a pharmaceutically acceptable salt thereof) that elicits the biological or medicinal response indicated. For example, an effective dose of a WEE1 inhibitor (including pharmaceutically acceptable salts thereof) (e.g., Azenosertib or a pharmaceutically acceptable salt thereof) can be the amount needed to prevent, alleviate or ameliorate symptoms of cancer, or prolong the survival of the subject being treated. This response may occur in a tissue, system, animal or human and includes alleviation of the signs or symptoms of the cancer being treated. Determination of an effective amount is well within the capability of those skilled in the art, in view of the disclosure provided herein. The effective dose of a WEE1 inhibitor (including pharmaceutically acceptable salts thereof) (e.g., Azenosertib or a pharmaceutically acceptable salt thereof) required as a dose will depend on the route of administration and the physical characteristics of the human subject under consideration. The effective dose can be tailored to achieve a desired effect, but will depend on such factors as weight, diet, concurrent medication and other factors which those skilled in the medical arts will recognize. In one embodiment, an effective dose of a WEE1 inhibitor (including pharmaceutically acceptable salts thereof) (e.g., Azenosertib or a pharmaceutically acceptable salt thereof) refers to an amount, including, but not limited to, a minimum amount, of Azenosertib, or a pharmaceutically acceptable salt thereof, that is sufficient to result in >10% (e.g., >15%, >20%, ≥25%, ≥30%, ≥35%, ≥40%) of a statistically significant population of subjects (e.g., at least 30 subjects) achieving an at least 18-week event-free survival (EFS) per RECIST v1.1 (i.e., time from treatment initiation until disease progression or death due to any cause).

In one embodiment, an effective dose of a WEE1 inhibitor (including pharmaceutically acceptable salts thereof) (e.g., Azenosertib, or a pharmaceutically acceptable salt thereof) refers to an amount, including, but not limited to, a minimum amount, of a WEE1 inhibitor (including pharmaceutically acceptable salts thereof) (e.g., Azenosertib, or a pharmaceutically acceptable salt thereof) that is sufficient to result in a statistically significant population of subjects (e.g., at least 30 subjects) achieving and maintaining a stable disease (SD) status for at least 16 weeks.

In one embodiment, an effective dose of a WEE1 inhibitor (including pharmaceutically acceptable salts thereof) (e.g., Azenosertib, or a pharmaceutically acceptable salt thereof) refers to an amount, including, but not limited to, a minimum amount, of a WEE1 inhibitor (including pharmaceutically acceptable salts thereof) (e.g., Azenosertib, or a pharmaceutically acceptable salt thereof) that is sufficient to result in a statistically significant population of subjects (e.g., at least 30 subjects) achieving a disease control rate (DCR) of ≥40% (e.g., ≥45%, ≥50%, ≥55%, ≥60%, ≥65%).

In some embodiments, the effective dose of a WEE1 inhibitor (including pharmaceutically acceptable salts thereof) (e.g., Azenosertib, or a pharmaceutically acceptable salt thereof) comprises a daily dose of at least about 250 mg, about 275 mg, about 300 mg, about 325 mg, about 350 mg, about 375 mg, about 400 mg, about 425 mg, about 450 mg, about 475 mg, about 500 mg, or an equivalent thereof, in one or more dosing weeks. In some embodiments, the effective dose of a WEE1 inhibitor (including pharmaceutically acceptable salts thereof) (e.g., Azenosertib, or a pharmaceutically acceptable salt thereof) comprises a daily dose of at least about 300 mg, about 325 mg, about 350 mg, about 375 mg, about 400 mg, about 425 mg, about 450 mg, or an equivalent thereof, in one or more dosing weeks. In some embodiments, the effective dose of a WEE1 inhibitor (including pharmaceutically acceptable salts thereof) (e.g., Azenosertib, or a pharmaceutically acceptable salt thereof) comprises a daily dose of at least about 300 mg, about 325 mg, about 350 mg, about 375 mg, about 400 mg, about 450 mg, about 500 mg, or an equivalent thereof, in one or more dosing weeks. In some embodiments, the effective dose of a WEE1 inhibitor (including pharmaceutically acceptable salts thereof) (e.g., Azenosertib, or a pharmaceutically acceptable salt thereof) comprises a daily dose of at least about 300 mg, about 350 mg, about 400 mg, about 450 mg, about 500 mg, or an equivalent thereof, in one or more dosing weeks. In some embodiments, the effective dose of a WEE1 inhibitor (including pharmaceutically acceptable salts thereof) (e.g., Azenosertib or a pharmaceutically acceptable salt thereof) comprises a daily dose of at least about 300 mg, or an equivalent thereof, in one or more dosing weeks. In some embodiments, the effective dose of a WEE1 inhibitor (including pharmaceutically acceptable salts thereof) (e.g., Azenosertib, or a pharmaceutically acceptable salt thereof) comprises a daily dose of at least about 350 mg, or an equivalent thereof, in one or more dosing weeks. In some embodiments, the effective dose of a WEE1 inhibitor (including pharmaceutically acceptable salts thereof) (e.g., Azenosertib, or a pharmaceutically acceptable salt thereof) comprises a daily dose of at least about 400 mg, or an equivalent thereof, in one or more dosing weeks. In some embodiments, the effective dose of a WEE1 inhibitor (including pharmaceutically acceptable salts thereof) (e.g., Azenosertib, or a pharmaceutically acceptable salt thereof) comprises a daily dose of at least about 450 mg, or an equivalent thereof, in one or more dosing weeks. In some embodiments, the effective dose of a WEE1 inhibitor (including pharmaceutically acceptable salts thereof) (e.g., Azenosertib, or a pharmaceutically acceptable salt thereof) comprises a daily dose of at least about 500 mg, or an equivalent thereof, in one or more dosing weeks.

In some embodiments, the effective dose of a WEE1 inhibitor (including pharmaceutically acceptable salts thereof) (e.g., Azenosertib, or a pharmaceutically acceptable salt thereof) comprises a daily dose of about 250 mg, about 275 mg, about 300 mg, about 325 mg, about 350 mg, about 375 mg, about 400 mg, about 425 mg, about 450 mg, about 475 mg, about 500 mg, or an equivalent thereof, in one or more dosing weeks. In some embodiments, the effective dose of a WEE1 inhibitor (including pharmaceutically acceptable salts thereof) (e.g., Azenosertib, or a pharmaceutically acceptable salt thereof) comprises a daily dose of about 300 mg, about 325 mg, about 350 mg, about 375 mg, about 400 mg, about 425 mg, about 450 mg, or an equivalent thereof, in one or more dosing weeks. In some embodiments, the effective dose of a WEE1 inhibitor (including pharmaceutically acceptable salts thereof) (e.g., Azenosertib, or a pharmaceutically acceptable salt thereof) comprises a daily dose of about 300 mg, about 325 mg, about 350 mg, about 375 mg, about 400 mg, or an equivalent thereof, in one or more dosing weeks. In some embodiments, the effective dose of a WEE1 inhibitor (including pharmaceutically acceptable salts thereof) (e.g., Azenosertib, or a pharmaceutically acceptable salt thereof) comprises a daily dose of about 300 mg, about 350 mg, about 400 mg, or an equivalent thereof, in one or more dosing weeks. In some embodiments, the effective dose of a WEE1 inhibitor (including pharmaceutically acceptable salts thereof) (e.g., Azenosertib, or a pharmaceutically acceptable salt thereof) comprises a daily dose of about 300 mg, or an equivalent thereof, in one or more dosing weeks. In some embodiments, the effective dose of a WEE1 inhibitor (including pharmaceutically acceptable salts thereof) (e.g., Azenosertib, or a pharmaceutically acceptable salt thereof) comprises a daily dose of about 350 mg, or an equivalent thereof, in one or more dosing weeks. In some embodiments, the effective dose of a WEE1 inhibitor (including pharmaceutically acceptable salts thereof) (e.g., Azenosertib, or a pharmaceutically acceptable salt thereof) comprises a daily dose of about 400 mg, or an equivalent thereof, in one or more dosing weeks. In some embodiments, the effective dose of a WEE1 inhibitor (including pharmaceutically acceptable salts thereof) (e.g., Azenosertib, or a pharmaceutically acceptable salt thereof) comprises a daily dose of about 450 mg, or an equivalent thereof, in one or more dosing weeks. In some embodiments, the effective dose of a WEE1 inhibitor (including pharmaceutically acceptable salts thereof) (e.g., Azenosertib, or a pharmaceutically acceptable salt thereof) comprises a daily dose of about 500 mg, or an equivalent thereof, in one or more dosing weeks.

In some embodiments of the methods described herein, the daily dose of WEE1 inhibitor (including pharmaceutically acceptable salts thereof) (e.g., Azenosertib, or a pharmaceutically acceptable salt thereof) is administered to the subject once a day (QD). In alternative embodiments, the daily dose of WEE1 inhibitor (including pharmaceutically acceptable salts thereof) (e.g., Azenosertib, or a pharmaceutically acceptable salt thereof) is administered to the subject twice a day (BID). The QD and/or BID dosing regimens may be varied from one dosing week to another.

In some embodiments of the methods described herein, the effective dose of a WEE1 inhibitor (including pharmaceutically acceptable salts thereof) (e.g., Azenosertib, or a pharmaceutically acceptable salt thereof) is provided on a continuous dosing schedule (e.g., 7 days a week with no intermission). In some embodiments of the methods described herein, the effective dose of a WEE1 inhibitor (including pharmaceutically acceptable salts thereof) (e.g., Azenosertib, or a pharmaceutically acceptable salt thereof) is provided on an intermittent dosing schedule, during which one or more dosing parameters, such as dosage amount and/or dosage interval are varied or changed. For example, an intermittent dosing phase may comprise a period of continuous administration (e.g., 6 days, 5 days, 4 days, 3 days, 2 days in a week) followed by a “rest” or intermission phase (e.g., 1 day, 2 days, 3 days, 4 days, 5 days in the same week) during which the WEE1 inhibitor (including pharmaceutically acceptable salts thereof) (e.g., Azenosertib, or a pharmaceutically acceptable salt thereof) is not administered or is administered at a reduced dosage amount and/or less frequently. A dosing regimen may further comprise one or more repeated cycles of intermittent dosing regimens. In one embodiment, the effective dose of a WEE1 inhibitor (including pharmaceutically acceptable salts thereof) (e.g., Azenosertib, or a pharmaceutically acceptable salt thereof) is provided on an intermittent dosing schedule comprising 5 days with dosing and 2 days without dosing in each of the one or more dosing weeks. In one embodiment, the effective dose of a WEE1 inhibitor (including pharmaceutically acceptable salts thereof) (e.g., Azenosertib, or a pharmaceutically acceptable salt thereof) is provided on an intermittent dosing schedule comprising 5 consecutive days with dosing and 2 consecutive days without dosing in each of the one or more dosing weeks. In one embodiment, the effective dose of a WEE1 inhibitor (including pharmaceutically acceptable salts thereof) (e.g., Azenosertib, or a pharmaceutically acceptable salt thereof) is provided on an intermittent dosing schedule comprising 4 days with dosing and 3 days without dosing in each of the one or more dosing weeks. In one embodiment, the effective dose of a WEE1 inhibitor (including pharmaceutically acceptable salts thereof) (e.g., Azenosertib, or a pharmaceutically acceptable salt thereof) is provided on an intermittent dosing schedule comprising 4 consecutive days with dosing and 3 consecutive days without dosing in each of the one or more dosing weeks. In one embodiment, the methods described herein include administering the effective dose of a WEE1 inhibitor on a 6 days on/1 day off, 5 days on/2 days off, or 4 days on/3 days off intermittent dosing schedule in one or more dosing weeks. In one embodiment, the methods described herein include administering the effective dose of a WEE1 inhibitor (including pharmaceutically acceptable salts thereof) (e.g., Azenosertib, or a pharmaceutically acceptable salt thereof) on a 5 days on/2 days off intermittent dosing schedule in one or more dosing weeks. In one embodiment, the methods described herein include administering the effective dose of a WEE1 inhibitor (including pharmaceutically acceptable salts thereof) (e.g., Azenosertib, or a pharmaceutically acceptable salt thereof) on a 4 days on/3 days off intermittent dosing schedule in one or more dosing weeks.

### EXAMPLES

Additional embodiments are disclosed in further detail in the following examples, which are not in any way intended to limit the scope of the claims.

### Example 1

Molecular Profiling of Subjects with Uterine Serous Carcinoma (USC)

Uterine serous carcinoma (USC) is an aggressive subtype of endometrial cancer characterized by ubiquitous TP53 mutations and high replication stress. The resulting dependency on DNA damage repair and cell cycle checkpoints triggers sensitivity to the abrogation of the G2/M checkpoint using WEE1 inhibitors, such as Azenosertib. WEE1 inhibitor sensitivity has been associated with high levels of Cyclin E1 expression, which may result from gene amplification of CCNE1 or the alteration of its ubiquitin ligase gene, FBXW7. In addition, signaling activity of WEE1 is controlled by the protein phosphatase 2A (PP2A), the regulatory subunit of which, PPP2R1A, is frequently mutated in USC.

The distribution and relationship of various potential oncogene and other cancer biomarker candidates to one another, including molecular alterations, was studied. Subjects with USC from the following sources were included: TCGA (UCEC/USC pan-cancer) (Levine et al., Nature (2013) 497:67-73); AACR-GENIE (v13, USC) (Consortium et al., Cancer Discov. (2017) 7 (8): 818-831); Foundation-Insights™ platform (20180129 FMI complete—USC); Caris Life Sciences Database (serous endometrial carcinoma), and USC subjects enrolled in Azenosertib clinical trials (with ClinicalTrials.gov IDs NCT04814108 and NCT04158336). This set of subjects was further restricted to those with pathogenic mutations in TP53 (70-95% of subjects) to ensure consistent histology.

The mutational status for all subjects in tumor DNA sequencing datasets were assessed for the following genes, known to be commonly altered (>10%) or of potential therapeutic interest in USC.
Pathogenic or likely pathogenic mutations in TP53, FBXW7, PPPR21A, PIK3CA, PIK32R1, or KRAS
Copy number (CN) amplifications of CCNE1, MYC, or ERBB2
Pathogenic or likely pathogenic mutations in at least one of the 14 homologous recombination and repair (HRR) genes: BRCA1, BRCA2, ATM, CHEK1, CHEK2, FANCL, CDK12, PALB2, BRIP1, BARD1, RAD51B, RAD51C, RAD51D, and RAD54L

The prevalence of molecular alterations in the genes identified above across all five different sources of patient data is shown in Table 1. Overall, the alteration prevalence is consistent across the databases and in the clinical trial cohorts, with gene amplification in CCNE1 and MYC being more frequent in the data obtained from TCGA.

TABLE 1
Prevalence of molecular alterations across five different sources

| | | | | | Azenosertib | |
|---|---|---|---|---|---|---|
| | | AACR- | | | clinical | |
| Molecular | TCGA | GENIE | Caris | FMI | trials | |
| alterations | (N = 95) | (N = 796) | (N = 3486) | (N = 685) | (N = 50) | Median |
| TP53m | 100% | 100% | 100% | 100% | 100% | 100% |
| PIK3CAm | 37% | 35% | 34% | 34% | 26% | 34% |
| PPP2R1Am | 37% | 30% | 26% | 29% | 35% | 30% |
| FBXW7m | 21% | 17% | 19% | 15% | 10% | 19% |
| PIK3R1m | 13% | 12% | 11% | 11% | 7% | 11% |
| KRASm | 4% | 5% | 4% | 6% | 4% | 4% |
| HRRm | 7% | 7% | 7% | 7% | 16% | 7% |
| BRCA2m | 1% | 2% | 2% | 2% | 4% | 2% |
| BRCA1m | 27% | 1% | 1% | 1% | 2% | 1% |
| CCNE1amp | 27% | 16% | 11% | 19% | 10% | 16% |
| ERBB2amp | 19% | 14% | 11% | 18% | 8% | 11% |
| MYCamp | 26% | 7% | 4% | 14% | 6% | 7% |

m: mutated; amp: amplified; HRRm: any pathogenic or likely pathogenic mutation in one or the 14 HRR genes.

### Example 2

### Identification of the PI3K-FBXW7-PPP2R1A Trio Molecular Combination and Subtypes and Characterization of the Same

Using the 8 most prevalent molecular alterations from Table 1 (not including TP53m because all subjects were already preselected for this mutation) from the four different sources, the molecular alterations were paired and subject to mutual exclusivity and co-mutation pattern analyses using Fisher-Exact test. Based on patterns of significant mutual exclusivity or co-mutation, three grouping schemes were used to investigate associations with, when available, other molecular features, selected protein expression, copy number signatures, selected signaling pathways, and clinical observations: (i) the combinations of FBXW7, PPP2R1A, and PI3K mutational statuses; (ii) HRR (mutant vs. wild-type); and (iii) (CCNE1 (amplified vs. non-amplified).

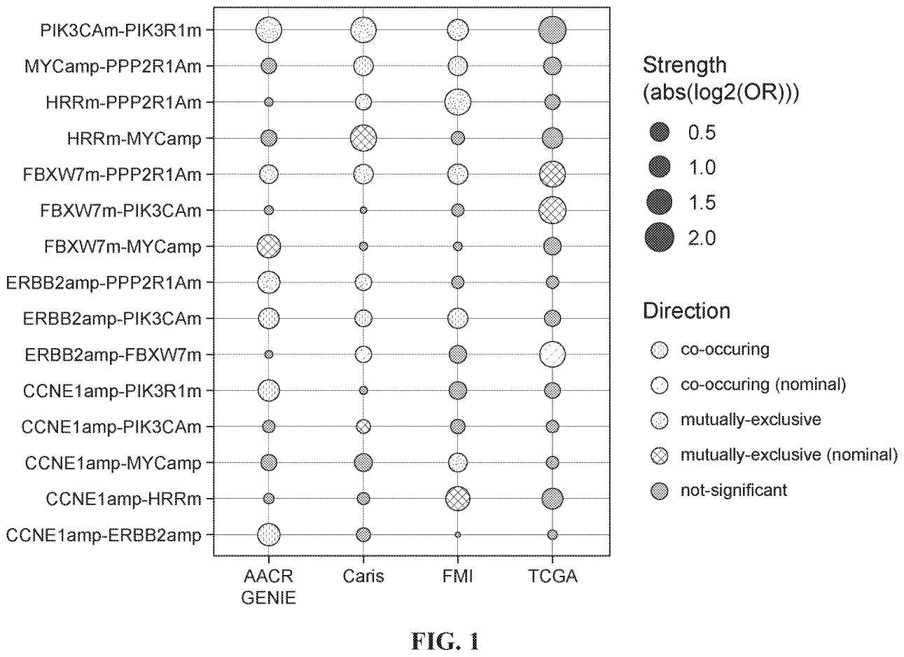

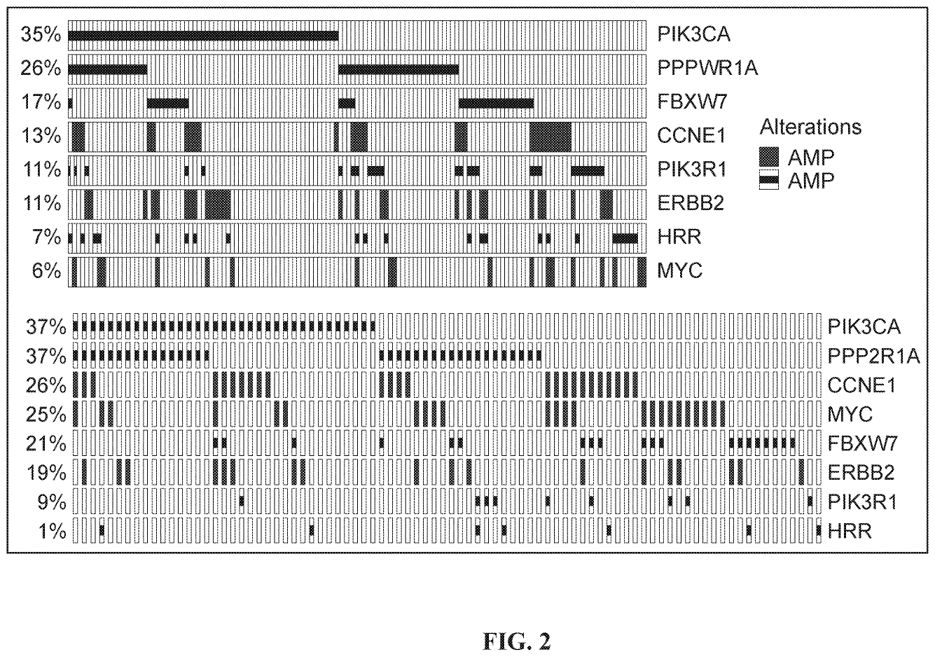

The analysis of the 15 molecular alteration pairs shown in FIG. 1 indicates that alterations in FBXW7 and PPP2R1A were consistently mutually exclusive and that phosphoinositide 3-kinase (PIK) was altered via mutually exclusive mutations in PIK3CA and PIK3R1. The oncoprint maps from AACR-GENIE ((N=796), top of FIG. 2) and TCGA ((N=95), bottom of FIG. 2) also indicate similar co-mutation and molecular exclusivity patterns.

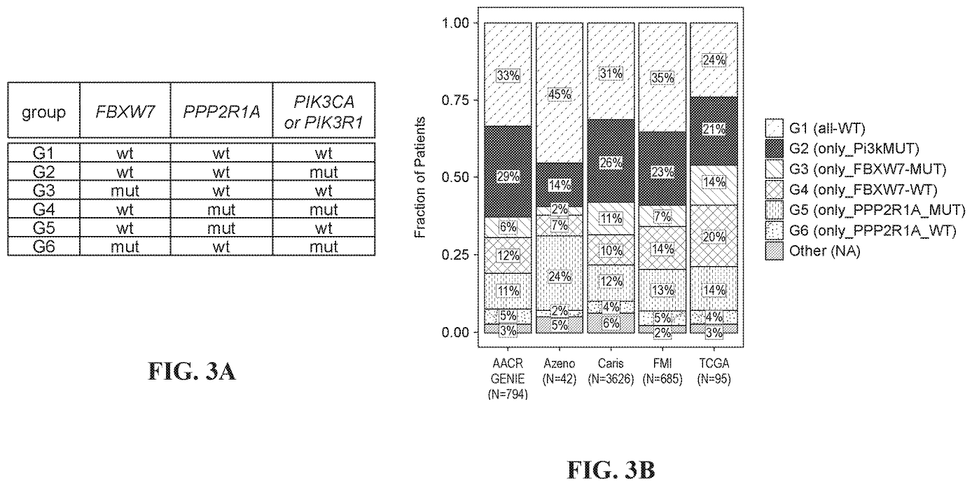

Based on the observed co-mutation and molecular exclusivity pattern analyses, the PI3K-FBXW7-PPP2R1A trio molecular combination was further studied for their prevalence from all five sources and divided into six distinct molecular subtypes, as shown in FIG. 3A, based on the mutational status of PIK3CA or PIK3R1, FBXW7, and PPP2R1A. Whilst, as discussed above, alterations in FBXW7 and PPP2R1A were mutually exclusive, alterations in FBXW7 and one of PIK3CA and PIK3R1 (i.e., group G6) and alterations in PPP2R1A and one of PIK3CA and PIK3R1 (i.e., group G4) were not mutually exclusive, with a prevalence of 2-5% and 7-20%, respectively (see FIG. 3B).

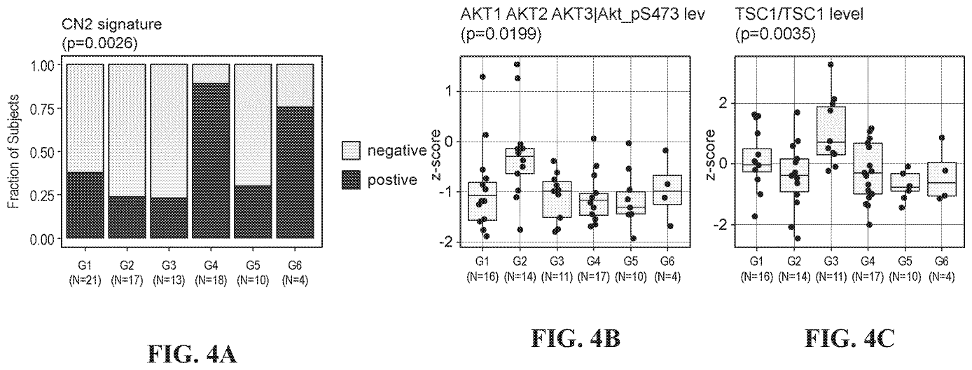

Among the TCGA cohort, alterations in FBXW7 and one of PIK3CA and PIK3R1 (i.e., group G6) and alterations in PPP2R1A and one of PIK3CA and PIK3R1 (i.e., group G4) are associated with the copy number signature CN2, which is a signature of a tetraploid genome that is indicative of genomic instability (see FIG. 4A; Steele et al., Nature (2022) 606:984-991). Referring to FIG. 4B (TCGA RPPA) and specifically comparing the molecular subtype G2 to subtypes G4 and G6 in the TCGA cohort, it is observed that mutations in FBXW7 or PPP2R1A reduce AKT (including AKT1, AKT2, and AKT3) signaling pathway in tumors with PI3K alterations, which suggests that mutations in FBXW7 or PPP2R1A modulate the consequences of PI3K alterations on PI3K/AKT/mTOR signaling. Furthermore, FIG. 4C (TCGA RPPA) indicates that mutations in FBXW7 in the absence of PI3K or PPP2R1A alterations (i.e., group G3) induce TSC1 levels. p-values in FIGS. 4A-4C were calculated using the Kruskal-Wallis test.

### Example 3

Association of HRR Mutational Status and CCNE1 Amplification Status with Overall Survival in Subjects with USC

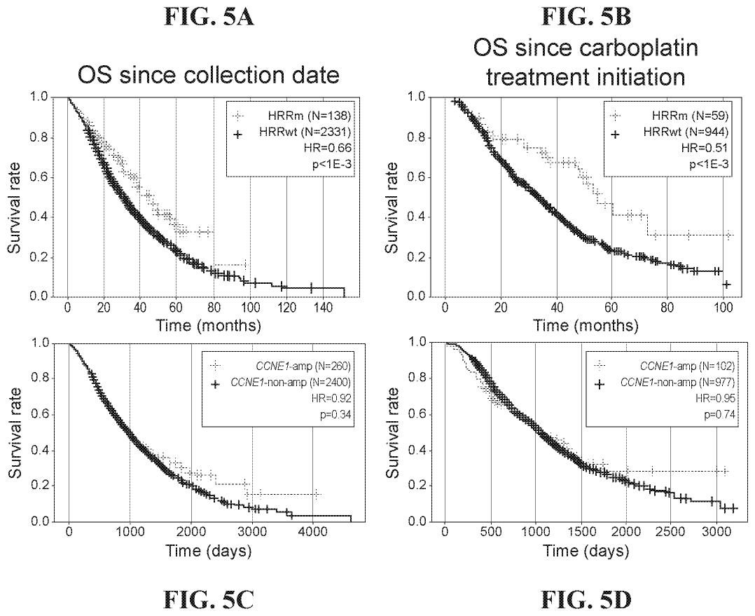

Overall survival (OS) analysis of HRRm and HRRwt or HRRm-negative subjects with USC from the Caris Life Science database (FIGS. 5A and 5B), CCNE1-amplified and non-CCNE1-amplified subjects with USC and unselected for TP53 status from the same database (FIGS. 5C and 5D) was performed. For FIGS. 5A and 5C, OS was measured since sample collection. For FIGS. 5B and 5D, OS was measured since the initiation of Carboplatin treatment. The OS analysis indicates that HRRm subjects had better overall survival while the CCNE1 amplification status was not prognostic.

### Example 4

Association of CCNE1 Amplification Status with Cyclin E1 Expression Levels in Subjects with USC

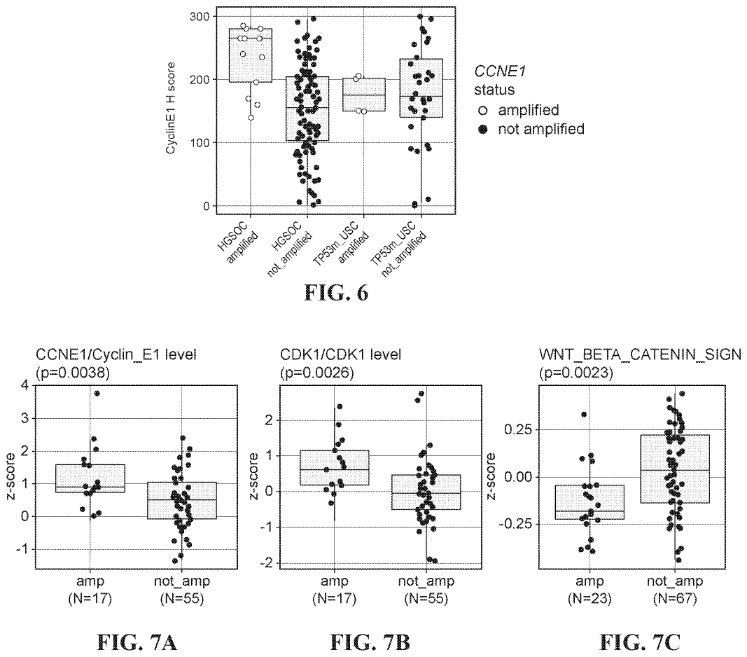

Subjects with TP53-mutated USC from Azenosertib clinical trials were examined for their Cyclin E1 expression and their immunohistochemistry (IHC) H-scores were calculated in accordance with the procedures described in Harismendy et al. (AACR Special Conference on Ovarian Cancer-Poster B035, (2023)). As shown in FIG. 6, regardless of CCNE1 amplification status, subjects with TP53-mutated USC and also high-grade serous ovarian cancer (HGSOC) can be associated with high expression levels of Cyclin E1, as indicated by their Cyclin E1 IHC H-scores.

Using the TCGA cohort instead, the CCNE1-amplified status is more strongly associated with high expression levels of Cyclin E1 (see FIG. 7A). Furthermore, the CCNE1-amplified status is also seen as being associated with high expression levels of CDK1 (FIG. 7B) and reduced Wnt/β-catenin signaling (FIG. 7C) from the same cohort.

### Example 5

Treatment of DLD-1 and DLD-1 FBXW7 Knockout (FBXW7-KO) Cancer Cells with WEE1 Inhibitor, Azenosertib

Parental DLD-1 colorectal cancer cells or FBXW7 knockout (FBXW7-KO) DLD-1 cells (Creative Biogene, Cat No. CSC-RT0018, Lot No. 081723ZYX) were cultured in RPMI 1640 medium (ATCC modification) (Giboco) supplemented with 10% FBS (Gibco) and 1% penicillin-streptomycin (Gibco). Cells were maintained at 37° C. in an atmosphere containing 5% CO₂.

The cultured cells were seeded at optimal density in 6-well plates, allowed to adhere overnight, and then treated with Azenosertib (250 nM, 1 μM, or 10 μM), with DMSO treatment as a negative control. After 8, 24, and 48 hours of drug exposure, cells were harvested, rinsed with PBS, lysed with cold RIPA buffer (Sigma) containing protease and phosphatase inhibitors for 15 minutes, and centrifuged at 4° C., 15,000 rpm for 15 minutes. Protein concentrations were measured using the Pierce BCA Protein Assay kit (Thermo Fisher). Jess™ plates were loaded and run according to the manufacturer's instructions (Bio-Techne). Primary antibodies anti-FBXW7 and anti-Cyclin E1 were purchased from Abcam, anti-phospho-H3, anti-Vinculin, anti-γH2AX, anti-phospho-CDK1, anti-phsopho-Chk1, anti-cleaved caspase 3, anti-Wee1, and anti-CDK1 were purchased from Cell Signaling Technology.

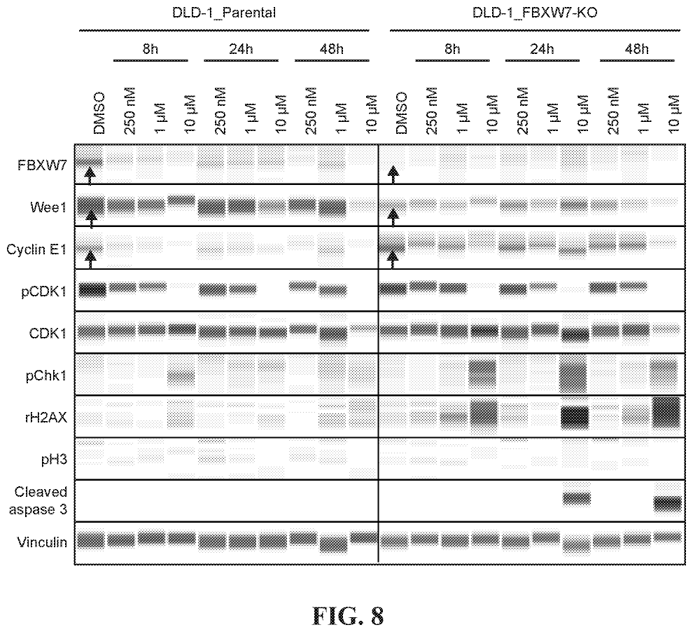

As shown in FIG. 8, the FBXW7-KO cells had lower levels of WEE1 kinase and higher levels of Cyclin E1 and Myc compared to the parental cell line to begin with, the latter observation suggesting higher replication stress. In both knockout cells and parental cells, treatment with Azenosertib led to dose-dependent decrease in WEE1 and phospho-CDK1 (pCDK1) levels, with the pCDK1 level decreasing significantly at 10 μM of Azenosertib and at 48 hours.

Of note, treatment with 10 μM Azenosertib led to more significant DNA damage in the knockout cells as compared to that in the parental cell line, as indicated by increase in phospho-Chk1 (pChk1) and gamma-H2AX (γH2AX) levels. Furthermore, treatment with Azenosertib (e.g., 10 UM at 24 hours and 48 hours) led to apoptosis in knockout cells as compared to that in the parental cell line, as indicated by increase in cleaved caspase 3 levels. These observations suggest that the FBXWY-KO cells were more sensitive to treatment with Azenosertib as compared to the parental cell line.

### Example 6

Alterations in FBXW7 are Associated with Better Clinical Outcomes in Cancer Patients Treated with Azenosertib Monotherapy

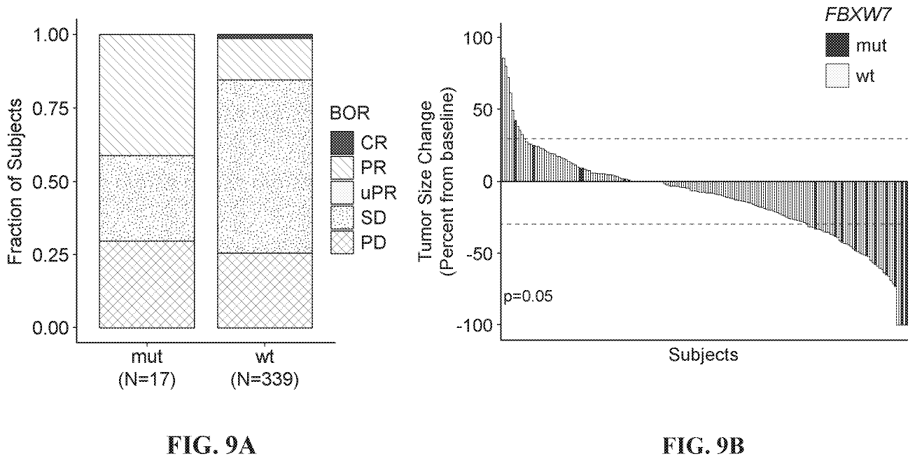

The predictive value of FBXW7 was investigated in a composite cohort of 356 patients treated with a daily dose of at least 300 mg of Azenosertib monotherapy on a continuous dosing schedule or an intermittent dosing schedule (e.g., 5 days on/2 days off, 4 days on/3 days off) and profiled with high-throughput DNA sequencing. Specifically, this composite cohort of patients was treated with a daily dose of 300 mg, or 350 mg, or 400 mg, or 450 mg of Azenosertib monotherapy on a continuous dosing schedule (i.e., QD, 7 days a week without any intermission), a daily dose of 300 mg, or 350 mg, or 400 mg, or 450 mg, or of 500 mg of Azenosertib monotherapy on an intermittent 5 days on/2 days off or 4 days on/3 days off schedule). The source of mutational status was a combination of local (reports from clinical sites) or central testing from either tumor or cell-free DNA. The mutational status was determined as mutated when at least one of the reports reported a pathogenic mutation and unmutated (wild-type) when all reports covering the FBXW7 gene were negative. The cohort investigated consists of patients diagnosed with high-grade serous ovarian carcinoma or HGSOC (N=211), uterine serous carcinoma or USC (N=57) or other solid tumor cancers including endometrial cancer, colorectal cancer, and peritoneum cancer (N=38). The patients mutated for FBXW7 (N=17-including 7 HGSOC/peritoneum, 7 USC, 2 endometrial and 3 colorectal), were more likely to respond to Azenosertib monotherapy (Objective Response Rate or ORR=41% (7/17) for FBXW7-mutated patients vs. 16% (53/339) for FBXW7-wild-type patients; p<0.02—FIG. 9A [BOR=Best Overall Response; CR=Complete Response; PR—Partial Response; uPR=Unconfirmed Partial Response; SD=Stable Disease; PD=Progressive Disease). Consistently, the target lesion from FBXW7-mutated patients showed greater size reduction (median −41% for FBXW7-wild-type patients vs. −8% for FBXW7-wild-type patients, p<0.05—FIG. 9B). These clinical observations suggest that FBXW7 mutations in solid tumor patients can predict better response to Azenosertib.

### EQUIVALENTS AND SCOPE

Those skilled in the art will recognize or be able to ascertain using no more than routine experimentation, many equivalents to the specific embodiments of the invention described herein. The scope of the present disclosure is not intended to be limited to the above Description, but rather is as set forth in the following claims.

1. A method of treating cancer comprising:
administering an effective dose of a WEE1 inhibitor to a subject selected to have a cancer associated with at least one alteration in the F-box and WD repeat domain containing 7 (FBXW7) gene.

2. A method of treating a cancer comprising:
administering an effective dose of a WEE1 inhibitor to a subject selected to have a cancer associated with at least one alteration in the F-box and WD repeat domain containing 7 (FBXW7) gene;
wherein:
the WEE1 inhibitor is Azenosertib, or a pharmaceutically acceptable salt thereof; and
the cancer is selected from colorectal cancer, peritoneum cancer, ovarian cancer and uterine cancer.

3. The method of claim 1 or claim 2, wherein the at least one alteration is selected from the group consisting of a nucleotide substitution, a nucleotide insertion and a nucleotide deletion.

4. The method of any one of claims 1 to 3, wherein the at least one alteration in the FBXW7 gene occurs at one or more nucleotides in a codon encoding an amino acid selected from the group consisting of E117, Q220, R224, R278, R367, G423, R441, R465, R479, R505, Y545, S582, R658, R668 and R689 in SEQ ID NO:1.

5. The method of any one of claims 1 to 3, wherein the at least one alteration in the FBXW7 gene is a splice-site mutation.

6. The method of any one of claims 1 to 5, wherein the at least one alteration in the FBXW7 gene results in an alternation selected from the group consisting of E117 deletion, Q220 frameshift mutation, (e.g., Q220fs*19), R465H, R479Q, R505C, R505G, R505L and R689W in SEQ ID NO: 1; and splice-site mutation FBXW7_c.585-4_592delins.

7. The method of any one of claims 1 to 6, wherein the subject is not further selected to have a cancer associated with any oncogene alteration or status, any oncoprotein expression level or status, or any signaling pathway level or alteration, other than the at least one alteration in FBXW7.

8. The method of any one of claims 1 and 3 to 7, wherein the WEE1 inhibitor is selected from the group consisting of Azenosertib (ZN-c3), Adavosertib (AZD1775), SC0191, Debio0123, IMP7068, PD0166285, NUV-569, SGR-3515, SY-4835, SPH-6162 April-1051 (formerly ATRN-W1051), ACR-2316, and a pharmaceutically acceptable salt of any of the foregoing.

9. The method of any one of claims 1 and 3 to 8, wherein the WEE1 inhibitor is Azenosertib (ZN-c3), or a pharmaceutically acceptable salt thereof.

10. The method of any one of claims 1 and 3 to 8, wherein the WEE1 inhibitor is Adavosertib (AZD1775), or a pharmaceutically acceptable salt thereof.

11. The method of any one of claims 1 and 3 to 8, wherein the WEE1 inhibitor is Debio0123, or a pharmaceutically acceptable salt thereof.

12. The method of any one of claims 1 and 3 to 8, wherein the WEE1 inhibitor is SGR-3515, or a pharmaceutically acceptable salt thereof.

13. The method of any one of claims 1 and 3 to 8, wherein the WEE1 inhibitor is APR-1051 (formerly ATRN-W1051), or a pharmaceutically acceptable salt thereof.

14. The method of any one of claims 1 and 3 to 8, wherein the WEE1 inhibitor is ACR-2316, or a pharmaceutically acceptable salt thereof.

15. The method of any one of claims 1 to 14, wherein the effective dose of a WEE1 inhibitor comprises a daily dose of at least about 250 mg, about 275 mg, about 300 mg, about 325 mg, about 350 mg, about 375 mg, about 400 mg, about 425 mg, about 450 mg, about 475 mg, about 500 mg, or an equivalent thereof, in one or more dosing weeks.

16. The method of any one of claims 1 to 15, wherein the effective dose of a WEE1 inhibitor comprises a daily dose of at least about 300 mg, about 325 mg, about 350 mg, about 375 mg, about 400 mg, about 425 mg, about 450 mg, about 500 mg, or an equivalent thereof, in one or more dosing weeks.

17. The method of any one of claims 1 to 16, wherein the effective dose of a WEE1 inhibitor comprises a daily dose of at least about 300 mg, about 350 mg, about 400 mg, about 450 mg, about 500 mg, or an equivalent thereof, in one or more dosing weeks.

18. The method of any one of claims 1 to 17, wherein the effective dose of a WEE1 inhibitor comprises a daily dose of at least about 300 mg or an equivalent thereof, in one or more dosing weeks.

19. The method of any one of claims 1 to 18, wherein the effective dose of a WEE1 inhibitor comprises a daily dose of at least about 350 mg or an equivalent thereof, in one or more dosing weeks.

20. The method of any one of claims 1 to 19, wherein the effective dose of a WEE1 inhibitor comprises a daily dose of at least about 400 mg or an equivalent thereof, in one or more dosing weeks.

21. The method of any one of claims 1 to 20, wherein the effective dose of a WEE1 inhibitor comprises a daily dose of at least about 450 mg or an equivalent thereof, in one or more dosing weeks.

22. The method of any one of claims 1 to 21, wherein the effective dose of a WEE1 inhibitor comprises a daily dose of at least about 500 mg or an equivalent thereof, in one or more dosing weeks.

23. The method of any one of claims 1 to 22, wherein the method comprises administering the effective dose of a WEE1 inhibitor on a continuous dosing schedule in one or more dosing weeks.

24. The method of any one of claims 1 to 23, wherein the method comprises administering the effective dose of a WEE1 inhibitor on an intermittent dosing schedule in one or more dosing weeks.

25. The method of claim 24, wherein the method comprises administering the effective dose of a WEE1 inhibitor on a 6 days on/1 day off, 5 days on/2 days off, or 4 days on/3 days off intermittent dosing schedule in one or more dosing weeks.

26. The method of claim 25, wherein the method comprises administering the effective dose of a WEE1 inhibitor 5 days on/2 days off intermittent dosing schedule in one or more dosing weeks.

27. The method of claim 25, wherein the method comprises administering the effective dose of a WEE1 inhibitor 4 days on/3 days off intermittent dosing schedule in one or more dosing weeks.

28. The method of any one of claims 1 and 3 to 27, wherein the cancer is selected from colorectal cancer, peritoneum cancer and a gynecologic malignancy.

29. The method of any one of claims 1 and 3 to 28, wherein the cancer is a gynecologic malignancy.

30. The method of claim 28 or claim 29, wherein the gynecologic malignancy is selected from cervical cancer, ovarian cancer, uterine cancer, vaginal cancer, vulvar cancer and fallopian tube cancer.

31. The method of any one of claims 1 to 30, wherein the cancer or the gynecologic malignancy is uterine cancer.

32. The method of any one of claims 1 to 31, wherein the cancer or the gynecologic malignancy is selected from endometrial cancer, endometrial endometrioid adenocarcinoma, uterine sarcoma, uterine serous carcinoma (USC) and uterine carcinosarcoma.

33. The method of any one of claims 1 to 32, wherein the cancer or the gynecologic malignancy is endometrial cancer.

34. The method of any one of claims 1 to 32, wherein the cancer or the gynecologic malignancy is uterine serous carcinoma (USC).

35. The method of any one of claims 1 to 30, wherein the cancer or the gynecologic malignancy is ovarian cancer.

36. The method of any one of claims 1 to 30 and 35, wherein the cancer or the gynecologic malignancy is selected from high-grade serous ovarian cancer (HGSOC), platinum-sensitive ovarian cancer, poly(ADP-ribose) polymerase inhibitor (PARPi)-resistant ovarian cancer, platinum-resistant ovarian cancer and platinum-refractory ovarian cancer.

37. The method of any one of claims 1 to 30, 35 and 36, wherein the cancer or the gynecologic malignancy is platinum-refractory ovarian cancer.

38. The method of any one of claims 1 to 30, 35 and 36, wherein the cancer or the gynecologic malignancy is high-grade serous ovarian cancer (HGSOC).

39. The method of any one of claims 1 to 28, wherein the cancer is colorectal cancer.

40. The method of any one of claims 1 to 28, wherein the cancer is peritoneum cancer.

41. The method of any one of claims 1 to 40, wherein the method comprises administering the effective dose of a WEE1 inhibitor as monotherapy.

42. The method of claim 40, wherein the method comprises administering the effective dose of a WEE1 inhibitor in combination with encorafenib, and optionally further in combination with cetuximab.

43. The method of any one of claims 1 to 42, wherein the subject has received no more than 1, at least 1, 1, 2, 3, 4, 1 or 2, 1 to 2, 1 to 3, or 1 to 4 prior line(s) of therapy, prior line(s) of therapy in the advanced or metastatic setting, prior line(s) of chemotherapy, prior line(s) of platinum-based chemotherapy, prior regimen(s), or prior therapeutic regimen(s).

44. The method of any one of claims 1 to 43, wherein the cancer is platinum-resistant, platinum-sensitive or platinum-refractory.

45. The method of any one of claims 1 to 44, wherein the cancer is PARP inhibitor-resistant.


SEQUENCE LISTING (1 sequence)

SEQ ID NO: 1 - protein, 707 aa, Homo sapiens
MNQELLSVGSKRRRTGGSLRGNPSSSQVDEEQMNRVVEEEQQQQLRQQEEEHTARNGEVVGVEPRPGGQNDSQQGQLEENNNRFISVDEDSSGNQEEQEEDEEHAGEQDEEDEEEEEMDQESDDFDQSDDSSREDEHTHTNSVTNSSSIVDLPVHQLSSPFYTKTTKMKRKLDHGSEVRSFSLGKKPCKVSEYTSTTGLVPCSATPTTFGDLRAANGQGQQRRRITSVQPPTGLQEWLKMFQSWSGPEKLLALDELIDSCEPTQVKHMMQVIEPQFQRDFISLLPKELALYVLSFLEPKDLLQAAQTCRYWRILAEDNLLWREKCKEEGIDEPLHIKRRKVIKPGFIHSPWKSAYIRQHRIDTNWRRGELKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFNKNFVITSSDDGTVKLWDLKTGEFIRNLVTLESGGSGGVVWRIRASNTKLVCAVGSRNGTEETKLLVLDFDVDMK